(19) United States
(12) Patent Application Publication
(10) Pub. No.: US 2026/0337578 A1
(43) Pub. Date: Oct. 29, 2026

(54) THERANOSTIC DEVICE FOR CANCER IMMUNOTHERAPY

(71) Applicants: ASPECT BIOSYSTEMS LTD., Vancouver (CA); The Royal Institution for the Advancement of Learning/McGill University, Montreal (CA)

(72) Inventors: Sam Wadsworth, Vancouver (CA); Erin Bedford, Vancouver (CA); Spiro Getsios, Nanaimo (CA); Tamer Mohamed, Richmond (CA); Konrad Walus, Vancouver (CA); Sheng Pan, Richmond (CA); Simon Beyer, Richmond (CA); Morag Park, Montreal (CA); Anne-Marie Norah Fortier, Montreal (CA)

(21) Appl. No.: 19/109,996

(22) PCT Filed: Sep. 7, 2023
(86) PCT No.: PCT/CA2023/051185

§ 371 Date: Mar. 7, 2025

Related U.S. Application Data
(60) Provisional application No. 63/404,517, filed on Sep. 7, 2022.

(51) Int. Cl. C12N 5/09; B33Y 80/00; G01N 33/50

(52) U.S. Cl. CPC C12N 5/0693; G01N 33/5011; B33Y 80/00; C12N 2503/04; C12N 2533/54

(57) ABSTRACT

Provided herein compositions and methods including bioprinted tissue fibers comprising precisely patterned cancerous and non-cancerous cells that are capable of recapitulating a desired tumor microenvironment in vitro. The subject tissue models enable the automated high throughput screening of anti-cancer therapies in a biologically-relevant human system, and can be further personalized with patient-specific cells.

### CROSS-REFERENCE TO RELATED APPLICATION

This application claims the benefit of priority to U.S. Provisional Application Ser. No. 63/404,517 filed on Sep. 7, 2022, the disclosure of which is expressly incorporated by reference herein in its entirety.

### FIELD OF THE INVENTION

The invention relates to compositions and methods for the fabrication and use of a three dimensional structure comprising precisely patterned regions that contain one or more different cell types (e.g., cancerous and non-cancerous cells) in such a manner as to enable screening of anti-cancer therapies in a biologically-relevant human system.

### BACKGROUND OF THE INVENTION

Malignant tumors (cancers) are the second leading cause of death in the United States, after heart disease (Boring et al., C A Cancel J. Clin. 43:7 (1993)). Cancer is characterized by the increase in the number of abnormal, or neoplastic, cells derived from a normal tissue that proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

Tumors typically grow as three-dimensional structures, and the way they interact with surrounding tissue is dependent on the architecture of the tumor. Previous studies have demonstrated that tumor cells grown as a 3D sphere (or “spheroid”) have very different sensitivity to drugs compared to the same cells grown in two dimensions, e.g., on plastic tissue culture plates.

Solid tumor growth is regulated by complex interactions of tumor cells with the tumor microenvironment (TME) including cancer associated fibroblasts (CAFs), various immune cells including tumor infiltrating lymphocytes (TILs) and extracellular matrix components (ECM) (Balkwill et al., J Cell Sci., 2012). Accurate modeling of the TME in vitro is challenging as there is significant genetic (Finak et al., Nat Med., 2008; Lehmann et al., J Clin Invest., 2011) and spatial (Gruosso et al., J Clin Invest., 2019) heterogeneity within TME components.

Certain tumor types are characterized by a lack or paucity of T-cell infiltration, resulting in so-called “cold tumors” that may adversely impact a patient's response to emerging immunotherapies including, e.g. immune checkpoint inhibitors. (Bonaventura et al., Frontiers in Immunology, 2019). For example, triple-negative breast cancer (TNBC) patients that have tumors with TILs restricted to the margins are considered immune-cold and typically have poor prognosis (Ali et al., Ann Oncol., 2014; Salgado et. al., Ann Oncol., 2015). Interfering with the mechanisms that exclude TILs from these tumors may improve the patient's response and outcome. Unfortunately, however, there are multiple mechanisms of tumor immune evasion.

As only a minority of cancer patients respond to current chemotherapies and immunotherapies, new technologies are required to enable drug developers to more accurately and efficiently interrogate the effect of novel therapeutics on the TME and improve patient outcome. While murine models have been extensively used, these are expensive, time consuming and may not be suitable for testing many human-specific immune-mediated therapeutics. Accordingly, a major barrier to the development and successful clinical implementation of cancer therapeutics is the availability of in vitro experimental models that accurately reflect the in vivo immune and tumor cell microenvironment along with its spatial complexity. The present invention addresses these and other unmet needs.

### SUMMARY OF INVENTION

The present invention addresses the foregoing needs in the art with the precise spatial patterning of multiple cell types via microfluidic bioprinting to generate tissue structures that recapitulate a multi-cellular TME both spatially and functionally. In particular, bioprinted tissue fibers are provided comprising a first region comprising a plurality of tumor cells, a second region comprising a plurality of stromal cells, and a third region comprising at least one immune cell population of interest. In an exemplary embodiment, the first region, the second region, and the third region are positioned adjacent to one another such that the second region forms an interface between the first and third regions, although other arrangements are also contemplated herein. The regions can be sequential regions positioned along the length of a fiber, and/or adjacent layers in a multi-layered core-shell fiber. In embodiments, at least one of the plurality of tumor cells, the plurality of stromal cells, and/or the immune cell population of interest are patient-specific.

In one aspect, the invention provides a bioprinted tissue fiber for evaluating immune cell infiltration of tumor cells, comprising a first region that comprises a plurality of tumor cells embedded in a first cross-linkable biocompatible material; a second region that comprises a plurality of stromal cells embedded in a second cross-linkable biocompatible material; and a third region that comprises at least one immune cell population of interest embedded in a third cross-linkable biocompatible material. In the exemplary embodiment illustrated in more detail herein, the first region, the second region, and the third region are positioned adjacent to one another such that the second region forms an interface between the first region and the third region.

In one embodiment, the first, second and third regions are positioned sequentially and adjacent to each other along the length of the fiber. In another embodiment, the bioprinted tissue fiber comprises a multi-layered core-shell fiber, wherein the first region is or comprises the solid core, the second region is or comprises at least one annulus layer, and the third region is or comprises at least one external shell layer.

In embodiments, the immune cell population of interest comprises one or more of T cells, B cells, NK cells, NKT cells, monocytes, macrophages, dendritic cells, CAR-T cells and/or CAR-NK cells. In embodiments, the plurality of tumor cells are solid tumor type, e.g. breast cancer (triple negative or not), prostate cancer, skin cancer, retinoblastoma, kidney cancer, lung cancer, bone cancer, bowel cancer, brain tumor (glioblastoma etc), pancreatic cancer, insulinoma, thyroid cancer, neuroblastoma, ovarian cancer, testicular cancer, and the like. In embodiments, the plurality of stromal cells comprises cancer-associated fibroblasts (CAF). In embodiments, at least one of the plurality of tumor cells, the plurality of CAF, and/or the immune cell population of interest are patient-specific.

In embodiments, the first, second and third cross-linkable biocompatible material are the same. In embodiments, the cross-linkable biocompatible material comprises between about .25% and 0.75% alginate, between about 0.4% and 0.6% alginate, or between about 0.5% and 0.6% alginate; at least about 0.5% alginate, at least about 0.55% alginate, at least about 0.5625% alginate, or 0.5625% alginate. In embodiments, the cross-linkable biocompatible material further comprises between about 1.0 mg/ml and 1.5 mg/ml collagen, or between about 1.1 and 1.3 mg/ml collagen; at least about 1.1 mg/ml collagen, at least about 1.2 mg/ml collagen, or 1.2 mg/ml collagen. In embodiments, the cross-linkable biocompatible material further comprises between about 15% and 35% of a basement membrane preparation such as Matrigel, Cultrex, Geltex, EHS Matrix Extract, or the like, or between about 20% and 30% of a basement membrane preparation, at least about 20% basement membrane preparation, at least about 25% basement membrane preparation, or 25% basement membrane preparation. In exemplary embodiments, the basement membrane preparation comprises Matrigel. In embodiments, one or more of the first, second and/or third cross-linkable biocompatible materials further comprises decellularized extracellular matrix and/or growth factors.

In embodiments, at least one cell type is labeled with a labeling reagent. In embodiments, each cell type is labeled with a different labeling reagent. In embodiments, the fiber is immobilized within a printed membrane that maintains the fiber in a single plane. In embodiments, the fiber is generated in a standard multi-well plate format.

In another aspect, the invention provides an in vitro model of tumor cell microenvironment, comprising at least one of the foregoing bioprinted tissue fibers.

In another aspect, the invention provides a customizable theranostic device for evaluating a cancer immunotherapy in a patient in need thereof, comprising at least one of the foregoing bioprinted tissue fibers, preferably wherein at least one of, at least two of, or each of the plurality of tumor cells, the plurality of CAF, and/or the at least one immune cell population of interest are patient-specific.

In a further aspect, the invention provides a method for evaluating the compatibility and/or efficacy of a candidate agent, comprising contacting at least one of the foregoing bioprinted tissue fibers with the candidate agent and detecting immune cell infiltration into the core of the fiber. In embodiments, the invention includes bioprinting the fiber with the subject candidate agent (e.g. an engineered immune cell such as a CAR-T cell or the like) added to at least one region, and preferably to the third region.

The invention also provides a method of screening a candidate agent for anti-cancer activity, comprising contacting at least one of the foregoing bioprinted tissue fibers with the candidate agent; and measuring an anti-cancer response in at least one cell type in the bioprinted tissue fiber; wherein an increase in the anti-cancer response in the presence of the candidate agent indicates that the candidate agent has anti-cancer activity.

In embodiments, the cells within the various regions may be distinctly labeled as described herein, and cell density may be varied as desired for the assay. Similarly, an extra-cellular matrix factor or soluble growth factor may be selectively included in a specific region of the bioprinted fiber. In embodiments, the bioprinted fiber may further comprise a cell-free region.

In embodiments, the synthetic tissue fiber structure is immobilized within a printed membrane that maintains the fiber in a single plane. In embodiments, the fiber is generated in a standard multi-well plate format.

In an exemplary embodiment, the invention provides an in vitro 3D tumor model that accurately reflects different immune subtypes of the Triple Negative Breast Cancer (TNBC) in vivo tumor microenvironment, including the spatial relationship between tumor, fibroblasts and TILs using tissue-matched tumor micro environment (TME) components.

In embodiments, the invention provides a synthetic tissue fiber structure comprising at least 5 different regions. In the depicted embodiment, a first region comprises cell type a (T-cells), a second, adjacent region comprises cell type b (stromal fibroblasts), and third region comprises cell type c (tumor cells). In one embodiment, the first region and the third region are each adjacent to a cell-free region.

In embodiments, the invention provides a synthetic tissue fiber structure comprising a first region containing stromal cells and a second, adjacent region containing cancer cells. The two regions can be positioned adjacent to one another, with an interface between the two regions. In some embodiments, over time the cancer cells may spread out of the second region and infiltrate one or more surrounding regions. Alternatively, in some embodiments, over time the cancer cells may shrink away from the interface between the first region and the second region. In some embodiments, over time the cancer cells may increase in density. Alternatively, in some embodiments, over time the cancer cells may decrease in density.

In embodiments, fluorescent signals from cells within a synthetic tissue fiber structure may change over time as a function of tumor response, e.g. a signal's intensity may increase over time with tumor growth or an increase in tumor volume, or may decrease over time with tumor cell death or a reduction in tumor volume.

In embodiments, the invention provides a synthetic tissue fiber structure deposited on the surface of a tissue culture plate in a pattern that comprises a plurality of turns, e.g., a repeating unit of five different regions, which can be separated by cell-free regions. In an exemplary embodiment, the repeating unit comprises, in order from the first end of the fiber towards the second end of the fiber, a first region comprising immune cells, a second region comprising tumor cells, a third region comprising stromal cells, a fourth region comprising tumor cells, and a fifth region comprising immune cells.

Other features, objects, and advantages will be apparent from the disclosure that follows.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A) Bioprinting using QUAD™ printhead. Valves allow switching between materials containing cancer cells (EPI) and fibroblasts (CAF) as depicted in the schema (top right panel). Immunofluorescence imaging shows pinching areas between materials (MDA-MB-231 cancer cells, in green; IMR-90 fibroblasts, in red) along the length of the fiber (bottom right panel). B) Bioprinting using CENTRA™ printhead. Different channels allow a core-shell fiber design with cancer cells and fibroblasts (EPI+CAF) in the core and immune cells (TIL) in the shell as depicted in the schema (top right panel). Immunofluorescence imaging requires z-stack 3D deconvolution to visualize material (MDA-MB-231 cancer cells, in green; IMR-90 fibroblasts, in red; Jurkat lymphocytes, in purple) from each compartment (bottom right panel). C) Bioprinting using TWO SHELL prototype printhead TP-153. Different channels allow a multi-layer core-two shell design with cancer cells (EPI) in the core, fibroblasts (CAF) within the inner shell and immune cells (TIL) within the outer shell as depicted by the schema (top right panel). Immunofluorescence imaging (MDA-MB-231 cancer cells, in green; IMR-90 fibroblasts, in red; Jurkat lymphocytes, in purple) shows that material patterning accurately recapitulates in vivo TME boundaries. D) Cell-free core-shell alginate-based fiber printed using CENTRA printhead with core material loaded with FITC-dextran. Different FITC-dextran sizes were used to model small molecule diffusion (3-5 kDa) and diffusion of larger antibody-based molecules (70 kDa FITC-dextran with a similar hydrodynamic radius to IgG). FITC-diffusion was assessed by fluorescent signal release in conditioned media (top panel) and real-time fluorescent imaging (bottom panel). E) Cancer cells (MDA-MB-231 cancer cells, in green) and fibroblasts (IMR-90 fibroblasts, in red) embedded in AGC-10TM BioInk (left panel) are encapsulated by the alginate gel which impairs migration and cell-cell interactions. Addition of 25% Matrigel® in the AGC-10TM BioInk (right panel) enable fibroblasts remodelling and cancer cell spreading.

FIG. 2A) Patient-derived TME components are fluorescent-labeled and bioprinted using TWO SHELL prototype printhead TP-153. B) Immunofluorescence imaging of the 3D-TNBC tissue (z projection of the core, top image; x projection, bottom image). C) 3D-TNBC tissue assay workflow.

FIG. 3. A) Confocal immunofluorescence of the 3D-TNBC tissue (GCRC2233-PDXO, in green; GCRC2233-CAF, in red; PBMC-derived T cells, in purple) at day of printing (Day 0, left panel) and after culturing for 3 days (middle panel) and 6 days (right panel). z projection of the core, top image; x projection, bottom image. Core is delineated by yellow lines, based on PDXOs x, y and z position. Scale bar: 70 μm. B) 3D image analysis showing number of surfaces detected from green channel fluorescent signal (GCRC2233-PDXO, left panel) and red channel fluorescent signal (GCRC2233-CAF, middle panel), and number of spots detected from far red channel fluorescent signal (T cells, right panel) using Imaris software. C) 3D image analysis showing ellipticity (left panel) and sphericity (right panel) scores detected from red channel fluorescent signal (GCRC2233-CAF) using Imaris software. D) Confocal immunofluorescence showing T cell infiltration into the tumor core (yellow lines) of the 3D-TNBC tissue (GCRC2233-PDXO, in green; GCRC2233-CAF, in red; PBMC-derived T cells, in purple) at day of printing (Day 0, top panel) and after culturing for 6 days (bottom panel). z projection of the core, top image; x projection, bottom image. Scale bar: 70 μm. E) Normalized position z of surfaces and spots detected. Dashed lines represent position z of core boundaries (from average of 15 replicates). F) 3D image analysis showing number of spots detected from far red channel fluorescent signal (T cells) inside the core of the bioprinted fiber (yellow lines).

FIG. 4. A) Confocal immunofluorescence of the 3D-TNBC tissue (GCRC2233-PDXO, in green; GCRC2233-CAF, in red; PBMC-derived T cells, in purple) after culturing for 6 days in presence of mock treatment (top panels), TGF-beta inhibitor A8301 (middle panels) or without CAF (bottom panels). z projection of the core, top image; x projection, bottom image. Core is delineated by yellow lines, based on PDXOs x, y and z position. Scale bar: 70 μm. B) Western blot of TGF-beta downstream signaling target phospho-SMAD2, collagen 1 alpha 1 and alpha-smooth muscle actin protein expression levels in GCRC2233-CAF exposed to TGF-beta and/or A8301. C) 3D image analysis showing number of surfaces detected from red channel fluorescent signal (GCRC2233-CAF) in presence of A8301 or mock treatment for 3 and 6 days. T-test p<0.05. D) 3D image analysis showing ellipticity (left panel) and sphericity (right panel) scores detected from red channel fluorescent signal (GCRC2233-CAF) in presence of A8301 or mock treatment for 3 and 6 days. E) Normalized position z of spots detected from far red channel fluorescent signal (T cells) in presence of mock treatment, A8301 inhibitor or without CAF for 3 and 6 days. Dashed lines represent position z of core boundaries (from average of 15 replicates). 3D image analysis showing number of spots detected from far red channel fluorescent signal (T cells) inside the core of the bioprinted fiber (yellow lines) (F) or in the full 3D-TNB C tissue (G) in presence of mock treatment, A8301 inhibitor or without CAF for 3 and 6 days. T-test p<0.05.

FIG. 5. A) Confocal immunofluorescence of the 3D-TNBC tissue (GCRC2243-PDXO, in green; GCRC2243-CAF, in red; PBMC-derived T cells, in purple; NucView® caspase-3 substrate, in white) after culturing for 6 days in presence of mock treatment (top panels) or TGF-beta inhibitor A8301 (bottom panels). Scale bar: 50 μm. B) 3D image analysis showing number of surfaces detected from green channel fluorescent signal (GCRC2243-PDXO) that colocalized with surfaces detected from blue channel fluorescent signal (Nuc View® caspase-3 substrate) in presence of mock treatment, A8301 or without CAF for 3 and 6 days. ANOVA test. C) 3D image analysis showing frequency of TILs in short distance from apoptotic PDXOs in presence of A8301 or mock treatment for 6 days. D) 3D modeling showing T cells (purple) that directly contact PDXOs (green). Scale bar: 15 μm.

FIG. 6. A) Confocal immunofluorescence of the 3D-TNBC tissue (GCRC2233-PDXO, in green; GCRC2233-CAF, in red; PBMC-derived T cells, in purple) after culturing for 3 days (top panels) in presence of IL-2 in media (middle panels) or bioink (bottom panels). Core is delineated by yellow lines. Scale bar: 70 μm. B) 3D image analysis showing number of spots detected from far red channel fluorescent signal (T cells, right panel). ANOVA test. C) 3D image analysis showing frequency of T cells in short distance from PDXOs in presence or not of IL-2 for 3 days.

### DETAILED DESCRIPTION

The present invention provides bioprinted fibers comprising adjacent regions of tumor cells, stromal cells and immune cells arranged such that they can interact in a physiologically-relevant manner, including 3D tumor spreading within a stroma and immune cell-mediated killing of 3D tumors. As demonstrated herein, different cell types within the fiber can be labelled with different fluorescent probes, and in this way, tumor cell proliferation, spreading and death can be monitored using standard fluorescent imaging techniques. Immune cell migration towards tumor cells can be measured by tracking the physical position of each independent fluorescent label of each cell type.

The regions of biological material may be approximately the same in terms of volume, or may be different. Cell density in different regions may be the same or different between different segments/compartments. In one embodiment, the first, second and third regions are positioned adjacent to one another along the length of the fiber. In one embodiment, a multi-layered core-shell fibers is provided wherein the first region is the core, the second region is one or more annulus layer(s), and the third region is one or more external layers.

In embodiments, patient-specific cells can be used in one or more regions. Using cells from resected patient tissue increases the clinical relevance of the bioprinted tissue, and also enables personalized anti-cancer theranostics to better predict a patient's response to therapy.

In embodiments, the cell-laden fiber can be immobilized within a thin transparent membrane to immobilize the fiber, making it suitable for automated imaging using a confocal fluorescent plate reader. Such an assay enables the high throughput screening of anti-cancer immuno-oncology therapies in a physiologically-relevant system with automated endpoint measurement.

Aspects of the model include a continuous fiber printed onto a transparent, permeable membrane using a micro-fluidic based printing system. The fiber is patterned into alternating regions of tumor cells, “normal” stromal fibroblasts (or other relevant cells for the particular tumor type). Immune cells (such as T cytotoxic cells or Natural killer cells) can optionally be included with or adjacent to the stromal compartment. The different cell types can be loaded pre-printing with fluorescent dyes of different excitation/emission spectra (such as Quantum Dots or Cell-tracker Green, Cell-tracker Blue, Cell Tracker Red).

The sequenced fiber is preferably immobilized on a cell-free transparent surface. In one embodiment, the fiber structure can be sandwiched between two thin 3D printed layers of hydrogel, each layer approximately 100 microns in thickness. It is possible to image the cells in the living tissues using confocal fluorescent microscopy; tumor growth and tumor cell migration can be tracked by the increase in intensity of fluorescent signal specific to the tumor cells, or by an increase in the volume of fiber positive for the tumor-specific fluorescent marker. Similarly, tumor killing by externally-applied drugs or via immune-cell activation can manifest as a reduction in tumor-specific fluorescent intensity or area. The ability to independently monitor the phenotype of each cell-type in the system is essential to identify any off-target effects of potential anti-cancer drugs on the other “normal” cells in the system. For example, in some embodiments, a therapy can kill tumor cells but can also selectively kill adjacent “normal” cells. This system allows the in-fiber sequencing of other cell types including liver, kidney, neuronal, lung, etc. in the same well to monitor off-target drug toxicity in other target organs. Similarly, the patterning of highly metabolic tissues, such as liver, into the fiber allows for monitoring the impact of drug metabolism on the efficacy or toxicity of the anti-cancer therapies.

The system can also be used to investigate and measure a variety of other cancer-relevant biological processes in live cultures. Direct interactions between cells of different types can be analyzed by measuring areas of overlapping fluorescent signals. Immune cell migration through tissues towards tumors can be modelled by analyzing the spreading of the immune cell-specific fluorescent signal along the fiber over time. Importantly all of these data can be continuously obtained from live cultures over the course of an experiment using automated confocal microscopy. In addition, the 3D tissue models can be bioprinted into multi-well plate format, enabling the automated measurement of fluorescent endpoints using automated confocal fluorescent plate reader technology.

The biocompatible matrix may be composed of a wide variety of natural or synthetic polymers that support the growth and viability of living cells. Examples of such materials include but are not limited to: Collagen, alginate, laminin, elastin, fibrin, hyaluronic acid, poly(ethylene) glycol based gels, gelatin, chitosan, agarose, peptides, or combinations thereof. Fiber diameters will typically range from 10 μm to 300 μm, while the fiber length can range anywhere from anywhere from 1 or 2 mm up to several metres in length. Cell density within the fiber is defined by the bioprinter and may be varied in a controlled way, with different cell or identical cell densities within each region as desired.

In some embodiments, a subject system can enable personalized medicine by accurately predicting suitable treatment. The use of patient-derived tumor cells will allow the system to be used as a high throughput physiologically-relevant patient-specific screen to examine which of a range of therapies will be effective in the individual.

Exemplary aspects of the invention include a bioprinted tissue that reproduces the spatial relationship between TNBC tumor cells, CAFs and TILs. Microfluidic 3D bioprinting was utilized to print a core-shell fiber design that recapitulates the immune-cold TME and permits monitoring of immune cell infiltration into the cancer cell core of the fiber. This innovative 3D-TNBC bioprinted tissue fiber enables phenotypic screening in a physiological human tissue to accurately identify novel anti-cancer molecules and immune-oncology therapeutics that may promote immune cell infiltration into the tumor core.

One advantage of the present invention is that, compared to 3D spheroids, different cell types can be programmatically sequenced into adjacent regions in a 3D fiber to precisely model the interface between tumor cells and the surrounding healthy stromal tissue including, e.g., so-called immune-cold tumor environments such as the 3D-TNBC fiber noted above.

Another advantage of the present invention is that each cell type can be pre-labelled with different non-toxic fluorescent markers such as quantum dots or cell tracker™ reagents to allow the independent monitoring of the proliferation and/or spreading of each cell type within the fiber.

Another advantage of the present invention is that relevant immune cells (e.g. T-cells) can be included in the fiber to model the interaction of immune cells with a 3D tumor.

Another advantage of the present invention is that any off-target effects of anti-cancer therapies on non-cancerous stromal cells in 3D (such as cell death) can be monitored in parallel with the effects on the tumor cells.

Another advantage of the present invention is that cells from other tissues can be loaded into adjacent compartments in the fiber to examine the off-target toxicity of potential cancer therapies in different organs in the same system.

Another advantage of the present invention is that cells from highly metabolic tissues can be incorporated into adjacent compartments in the fiber to monitor how metabolism of the drug by tissues such as the liver, will impact the efficacy of the drug therapy.

Another advantage of the present invention is that all cells in the system can be added at different densities to precisely recapitulate the relevant tissue physiology.

Another advantage of the present invention is that disease-relevant extracellular matrix factors and soluble growth factors can be selectively included in different compartments of the fiber to precisely recapitulate the non-cellular environment of the tumor and stromal tissue.

Another advantage of the present invention is that small molecules, antibodies, antisense oligonucleotides, modified immune cells or other drug therapies can be selectively added to different compartments of the fiber to allow precise localization of a drug within the system.

Another advantage of the present invention is that the sequenced fiber is immobilized within a thin transparent printed membrane to maintain the fiber in a single plane. Cells incorporated into the fiber are loaded with non-toxic fluorescent dyes (such as CellTracker) to enable imaging using automated fluorescent microscopy.

Another advantage of the present invention is that fibers can be generated in standard multi-well plate formats (such as 96-well plates), making the tissue assay suitable for automated compound screening using cell-specific fluorescence read by automated plate based confocal instruments as an output.

Another advantage of the present invention is that multiple units of cell-containing regions (e.g., multiple units containing “immune cell-normal cell-tumor cell” regions) can be printed into a single fiber in each well, so multiple data-points on anti-cancer therapy effectiveness can be measured in a single well, further increasing throughput of the system.

Another advantage of the present invention is that the tumor regions of the fiber can be arranged in a consistent pattern to avoid any contribution of fiber shape to the assay.

### DEFINITIONS

For purposes of interpreting this specification, the following definitions will apply, and whenever appropriate, terms used in the singular will also include the plural and vice versa. In the event that any definition set forth conflicts with any document incorporated herein by reference, the definition set forth below shall control.

The term “region” as used herein refers to distinct layers, segments, or compartments of bioprinted tissue fiber that contain the same or different biomaterials, cells, and growth factors. The regions of biomaterial and cells may, in some embodiments, be approximately the same in terms of length and/or thickness, or may be different. Cell type and density in different regions of a fiber may be the same or different.

As used herein when referring to a “cell”, “cell line”, “cell culture” or “cell population” or “population of cells”, the term “isolated” refers to being substantially separated from the source of the cells such that the living cell, cell line, cell culture, cell population or population of cells are capable of being cultured in vitro for extended periods of time. In addition, the term “isolating” can be used to refer to the physical selection of one or more cells out of a group of two or more cells, wherein the cells are selected based on cell morphology and/or the expression of various markers.

The term “solidified” as used herein refers to a solid or semi-solid state of material that maintains its shape fidelity and structural integrity upon deposition. The term “shape fidelity” as used herein means the ability of a material to maintain its three dimensional shape without significant spreading. In some embodiments, a solidified material is one having the ability to maintain its three dimensional shape for a period of time of about 30 seconds or more, such as about 1, 10 or 30 minutes or more, such as about 1, 10, 24, or 48 hours or more. The term “structural integrity” as used herein means the ability of a material to hold together under a load, including its own weight, while resisting breakage or bending.

In some embodiments, a solidified composition is one having an elastic modulus greater than about 5, 10, 15, 20 or 25 kilopascals (kPa), more preferably greater than about 30, 40, 50, 60, 70, 80 or 90 kPa, still more preferably greater than about 100, 110, 120 or 130 kPa. Preferred elastic modulus ranges include from about 5, 10, 15, 20, 25 or 50 Pa to about 80, 100, 120 or 140 kPa. According to the subject invention, the elastic modulus of an input material can be advantageously varied according to the intended function of the input material. In some embodiments, a lower elastic modulus is employed to support cell growth and migration, while in other embodiments, a much higher elastic modulus can be used. In some embodiments, the elastic modulus may vary between different layers within a fiber.

The term “hydrogel” as used herein refers to a composition comprising water and a network or lattice of polymer chains that are hydrophilic.

The term “solid core” as used herein refers to a core of a fiber of the present disclosure that is comprised of a particular material (e.g., hydrogel cross-linkable by a chemical cross-linking agent), such that the core does not comprise a lumen along the entire length of the fiber. The term is not intended to refer to a core that is entirely impenetrable along its length, as solid cores of the present disclosure may enable the passage of particular fluids, molecules and/or ionic species throughout the core.

The term “annulus fibers” as used herein refer to fibers that are comprised of a solid core, and one or more shell layers surrounding the solid core. In embodiments, the core of an annulus fiber is surrounded by a first inner shell, and by a second outer shell, although fibers with greater numbers of shells (e.g., three, four, five, or more) are included within the definition of an annulus fiber of the present disclosure.

The term “biocompatible materials” as used herein refer to materials in which biological materials including but not limited to cells can be incorporated into and/or in contact with said biocompatible materials and where said biocompatible materials do not exhibit an adverse effect on the ability of the biological materials to carry out one or more functions (e.g., cellular functions including but not limited to secretion of biologically relevant molecular species, agonist/receptor binding, signal transduction, and the like). Examples of biocompatible materials as herein disclosed can include but are not limited to alginate, functionalized alginate (e.g. RGD-alginate, YIGSR-alginate), collagen, collagen-1, basement membrane proteins, collagen-4, collagen-2, fibronectin, fibrin, gelatin, vitronectin, laminin, decellularized extracellular matrices (dECM), hyaluronic acid (HA), polyethylene glycol (PEG), PEGDA and other functionalized PEG, fibrin, gelatin, gelatin-methacryloyl (GEL-MA), silk, chitosan, cellulose, polyoligo (ethylene glycol) methyl ether methacrylate (POEGMA), self assembling peptide hydrogels, or a combination thereof.

The term “functionalized alginate” as used herein refers to alginate that is chemically modified to include one or more properties that are advantageous in the manufacture of a fiber of the present disclosure. Examples of functionalized alginates include but are not limited to methacrylated alginate, alginate furan, alginate thiol, alginate maleimide, RGD-alginate, YIGSR-alginate, and covalent click alginates (e.g., alginate blended with DMAPS-Ald and/or DMAPS-Hzd).

The term “agent” as used herein refers to any protein, nucleic acid molecule (including chemically modified nucleic acid molecules), antibody, small molecule, organic compound, inorganic compound, or other molecule of interest. An agent can include a biologically relevant agent, a therapeutic agent, a diagnostic agent or a pharmaceutical agent. A therapeutic or pharmaceutical agent is one that alone or together with an additional compound induces a desired response (such as inducing a therapeutic or prophylactic effect when administered in a manner consistent with the present disclosure to a subject. A biologically relevant agent is one that supports another biological process, for example an agent that supports cell viability.

### Input Materials:

Aspects of the invention include input materials that can be used for printing fiber structures. In some embodiments, an input material comprises a hydrogel. Non-limiting examples of hydrogels include alginate, agarose, collagen, fibrinogen, gelatin, chitosan, hyaluronic acid based gels, or any combination thereof. A variety of synthetic hydrogels are known and can be used in embodiments of the systems and methods provided herein. For example, in some embodiments, one or more hydrogels form at least part of the structural basis for three dimensional structures that are printed. In some embodiments, a hydrogel has the capacity to support growth and/or proliferation of one or more cell types, which may be dispersed within the hydrogel or added to the hydrogel after it has been printed in a three dimensional configuration. In some embodiments, a hydrogel is cross-linkable by a chemical cross-linking agent. For example, a hydrogel comprising alginate may be cross-linkable in the presence of a divalent cation, a hydrogel containing chitosan may be cross-linked using a polyvalent anion such as sodium tripolyphosphate (STP), a hydrogel comprising fibrinogen may be cross-linkable in the presence of an enzyme such as thrombin, and a hydrogel comprising collagen, gelatin, agarose or chitosan may be cross-linkable in the presence of heat or a basic solution. In some embodiments hydrogel fibers may be generated through a precipitation reaction achieved via solvent extraction from the input material upon exposure to a cross-linker material that is miscible with the input material. Non-limiting examples of input materials that form fibers via a precipitation reaction include collagen and polylactic acid (PLA). Non-limiting examples of cross-linking materials that enable precipitation-mediated hydrogel fiber formation include polyethylene glycol (PEG) and alginate. Cross-linking of the hydrogel will increase the hardness of the hydrogel, in some embodiments allowing formation of a solidified hydrogel.

In some embodiments, a hydrogel comprises alginate. Alginate forms solidified colloidal gels (high water content gels, or hydrogels) when contacted with divalent cations. Any suitable divalent cation can be used to form a solidified hydrogel with an input material that comprises alginate. In the alginate ion affinity series Cd²⁺>Ba²⁺>Cu²⁺>Ca²⁺>Ni²⁺>Co²⁺>Mn²⁺, Ca²⁺ is the best characterized and most used to form alginate gels (Ouwerx, C. et al., Polymer Gels and Networks, 1998, 6 (5): 393-408). Studies indicate that Ca-alginate gels form via a cooperative binding of Ca²⁺ ions by poly G blocks on adjacent polymer chains, the so-called “egg-box” model (ISP Alginates, Section 3: Algin-Manufacture and Structure, in Alginates: Products for Scientific Water Control, 2000, International Specialty Products: San Diego, pp. 4-7). G-rich alginates tend to form thermally stable, strong yet brittle Ca-gels, while M-rich alginates tend to form less thermally stable, weaker but more elastic gels. In some embodiments, a hydrogel comprises a depolymerized alginate.

In some embodiments, a hydrogel is cross-linkable using a free-radical polymerization reaction to generate covalent bonds between molecules. Free radicals can be generated by exposing a photoinitiator to light (often ultraviolet), or by exposing the hydrogel precursor to a chemical source of free radicals such as ammonium peroxodisulfate (APS) or potassium peroxodisulfate (KPS) in combination with N,N,N,N-Tetramethylethylenediamine (TEMED) as the initiator and catalyst respectively. Non-limiting examples of photo cross-linkable hydrogels include: methacrylated hydrogels, such as gelatin methacrylate (GEL-MA) or polyethylene (glycol) acrylate-based (PEG-Acylate) hydrogels, which are used in cell biology due to their ability to cross-link in presence of free radicals after exposure to UV light and due to their inertness to cells. Polyethylene glycol diacrylate (PEG-DA) is commonly used as scaffold in tissue engineering, since polymerization occurs rapidly at room temperature and requires low energy input, has high water content, is elastic, and can be customized to include a variety of biological molecules.

Input materials in accordance with embodiments of the invention can comprise any of a wide variety of natural or synthetic polymers that support the viability of living cells, including, e.g., alginate, laminin, fibrin, hyaluronic acid, poly(ethylene) glycol based gels, gelatin, chitosan, agarose, or combinations thereof. In some embodiments, the subject bioink compositions are physiologically compatible, i.e., conducive to cell growth, differentiation and communication. In certain embodiments, an input material comprises one or more physiological matrix materials, or a combination thereof. By “physiological matrix material” is meant a biological material found in a native mammalian tissue. Non-limiting examples of such physiological matrix materials include: fibronectin, thrombospondin, glycosaminoglycans (GAG) (e.g., hyaluronic acid, chondroitin-6-sulfate, dermatan sulfate, chondroitin-4-sulfate, or keratin sulfate), deoxyribonucleic acid (DNA), adhesion glycoproteins, and collagen (e.g., collagen I, collagen II, collagen III, collagen IV, collagen V, collagen VI, or collagen XVIII).

In embodiments, the first, second and third cross-linkable biocompatible materials are the same. In preferred embodiments, the cross-linkable biocompatible material comprises between about .25% and 0.75% alginate, between about 0.4% and 0.6% alginate, or between about 0.5% and 0.6% alginate; at least about 0.5% alginate, at least about 0.55% alginate, at least about 0.5625% alginate, or 0.5625% alginate. In embodiments, the cross-linkable biocompatible material further comprises between about 1 mg/ml and 1.5 mg/ml collagen, or between about 1.1 and 1.3 mg/ml collagen; at least about 1.1 mg/ml collagen, at least about 1.2 mg/ml collagen, or 1.2 mg/ml collagen. In embodiments, the cross-linkable biocompatible material further comprises between about 15% and 35% of a basement membrane extract (BME) such as Matrigel, Cultrex, Geltex, or the like, or between about 20% and 30% BME; at least about 20% BME, at least about 25% BME, or 25% BME. In exemplary embodiments, the basement membrane extract comprises Matrigel. In embodiments, one or more of the first, second and/or third cross-linkable biocompatible materials further comprises decellularized extracellular matrix and/or growth factors.

### Cell Populations

Input materials in accordance with embodiments of the invention can incorporate any mammalian cell type, including but not limited to stem cells (e.g., embryonic stem cells, adult stem cells, induced pluripotent stem cells), germ cells, endoderm cells (e.g., lung, liver, pancreas, gastrointestinal tract, or urogenital tract cells), mesoderm cells (e.g., kidney, bone, muscle, endothelial, or heart cells), ectoderm cells (skin, nervous system, pituitary, or eye cells), stem cell-derived cells, and tumor cells, or any combination thereof.

In embodiments, the first region of the bioprinted fiber of the subject invention comprises a plurality of tumor cells embedded in a first cross-linkable biocompatible material; the second region comprises a plurality of stromal cells embedded in a second cross-linkable biocompatible material; and the third region comprises at least one immune cell population of interest embedded in a third cross-linkable biocompatible material. In embodiments, the immune cell population of interest comprises one or more of T cells, B cells, NK cells, NKT cells, monocytes, macrophages, dendritic cells, CAR-T cells and/or CAR-NK cells. In embodiments, the plurality of tumor cells are solid tumor type, e.g. breast cancer (triple negative or not), prostate cancer, skin cancer, retinoblastoma, kidney cancer, lung cancer, bone cancer, bowel cancer, brain tumor (glioblastoma etc), pancreatic cancer, insulinoma, thyroid cancer, neuroblastoma, ovarian cancer, testicular cancer, and the like. In embodiments, the plurality of stromal cells comprises cancer-associated fibroblasts (CAF).

In an exemplary embodiment, the first region, the second region, and the third region are positioned adjacent to one another within the fiber such that the second region forms an interface between the first and third regions. In one such embodiment, the bioprinted fiber comprises a multi-layered core/shell/shell architecture, with the core comprising the first region, the annulus layer comprising the second region, and the outer shell layer comprising the third region.

As the skilled artisan will readily appreciate, however, and notwithstanding the foregoing exemplified embodiments, the relative positioning of the regions and their respective cell populations can be altered within the fiber to replicate different biological environments and/or to investigate different biological effects. As but one example, the ordering designated above can be reversed such that the first region comprising the tumor cells is positioned in the outside shell layer of a multi-layered fiber rather than the internal core, to study and/or isolate potential issues of hypoxia on tumor cells. In another example, the second region comprising the stromal cells can be positioned in the outside shell layer of a multi-layered fiber relative to the tumor cells and immune cells, to study and/or isolate the effect of CAF-secreted factors on immune cell activity and/or migration including killing of the tumor cells.

In an alternative embodiment, therefore, the first region, the second region, and the third region are positioned adjacent to one another within the fiber such that the second region forms an interface between the first and third regions, but in this embodiment the bioprinted fiber comprises a multi-layered core/shell/shell architecture with the core comprising the third region, the annulus layer comprising the second region, and the outer shell layer comprising the first region. In another alternative embodiment, the first region, the second region, and the third region are positioned adjacent to one another within the fiber such that the first region forms an interface between the second and third regions, or the third region forms an interface between the first and second regions. In one such embodiment, the bioprinted fiber comprises a multi-layered core/shell/shell architecture with the core comprising the first or third region, the annulus layer comprising the third or first region, and the outer shell layer comprising the second region. Additional advantageous embodiments may be readily envisaged by the skilled artisan based on the above.

In embodiments, at least one of the plurality of tumor cells, the plurality of CAF, and/or the immune cell population of interest are patient-specific, i.e. derived from a patient undergoing evaluation for and/or treatment of cancer, e.g. a cancer immunotherapy.

Appropriate growth conditions for mammalian cells are well known in the art (Freshney, R. I. (2000) Culture of Animal Cells, a Manual of Basic Technique. Hoboken N.J., John Wiley & Sons; Lanza et al. Principles of Tissue Engineering, Academic Press; 2nd edition May 15, 2000; and Lanza & Atala, Methods of Tissue Engineering Academic Press; 1st edition October 2001). Cell culture media generally include essential nutrients and, optionally, additional elements such as growth factors, salts, minerals, vitamins, etc., that may be selected according to the cell type(s) being cultured. Particular ingredients may be selected to enhance cell growth, differentiation, secretion of specific proteins, etc. In general, standard growth media include Dulbecco's Modified Eagle Medium, low glucose (DMEM), with 110 mg/L pyruvate and glutamine, supplemented with 10-20% fetal bovine serum (FBS) or calf serum and 100 U/ml penicillin are appropriate as are various other standard media well known to those in the art. Growth conditions will vary depending on the type of mammalian cells in use and the tissue desired.

In some embodiments, cell-type specific reagents can be advantageously employed in the subject input materials for use with a corresponding cell type. For example, a decellularized extracellular matrix (“ECM”) can be extracted directly from a tissue of interest and then solubilized and incorporated it into an input material to generate tissue-specific input materials for printed tissues. Such ECMs can be readily obtained from patient samples and/or are available commercially from suppliers such as zPredicta (rBone™, available at zpredicta.com/home/products)

### Active Agents:

In some aspects, embodiments of the invention can comprise at least one active agent added to fibers of the present disclosure during printing, e.g. biologically relevant agents to help facilitate cell growth and/or differentiation. Non-limiting examples of such active agents include TGF-β1, TGF-β2, TGF-β3, BMP-2, BMP-4, BMP-6, BMP-12, BMP-13, basic fibroblast growth factor, fibroblast growth factor-1, fibroblast growth factor-2, platelet-derived growth factor-AA, platelet-derived growth factor-BB, platelet rich plasma, IGF-I, IGF-II, GDF-5, GDF-6, GDF-8, GDF-10, vascular endothelial cell-derived growth factor, pleiotrophin, endothelin, nicotinamide, glucagon like peptide-I, glucagon like peptide-II, parathyroid hormone, tenascin-C, tropoelastin, thrombin-derived peptides, laminin, biological peptides containing cell-binding domains and biological peptides containing heparin-binding domains, therapeutic agents, and any combinations thereof.

Additional active agents can include, but are not limited to, proteins, peptides, nucleic acid analogues, nucleotides, oligonucleotides, nucleic acids (DNA, RNA, siRNA, mRNA), peptide nucleic acids, aptamers, antibodies or fragments or portions thereof, antigens or epitopes, hormones, hormone antagonists, growth factors or recombinant growth factors and fragments and variants thereof, cytokines, enzymes, antibiotics or antimicrobial compounds, anti-inflammation agent, antifungals, antivirals, toxins, prodrugs, small molecules, drugs (e.g., drugs, dyes, amino acids, vitamins, antioxidants) or any combination thereof.

Non-limiting examples of anti-inflammatory and anti-fibrotic factors that are suitable for inclusion as an input material include: steroids (dexamethasone), pirfenidone, prostaglandin agonists (butaprost), rapamycin, GW2580, and the like.

Non-limiting examples of antibiotics that are suitable for inclusion in an input material include: aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, or any combination thereof.

Non-limiting examples of antibodies include: abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, zanolimumab, or any combination thereof.

Non-limiting examples of enzymes suitable for use in an input material as described herein include: peroxidase, lipase, amylose, organophosphate dehydrogenase, ligases, restriction endonucleases, ribonucleases, DNA polymerases, glucose oxidase, and laccase.

Additional non-limiting examples of active agents that are suitable for use with the subject input materials include: cell growth media, such as Dulbecco's Modified Eagle Medium, fetal bovine serum, non-essential amino acids and antibiotics; growth and morphogenic factors such as fibroblast growth factor, transforming growth factors, vascular endothelial growth factor, epidermal growth factor, platelet derived growth factor, insulin-like growth factors), bone morphogenetic growth factors, bone morphogenetic-like proteins, transforming growth factors, nerve growth factors, and related proteins (growth factors are known in the art, see, e.g., Rosen & Thies, CELLULAR & MOLECULAR BASIS BONE FORMATION & REPAIR (R. G. Landes Co., Austin, Tex., 1995); anti-angiogenic proteins such as endostatin, and other naturally derived or genetically engineered proteins; polysaccharides, glycoproteins, or lipoproteins; anti-infectives such as antibiotics and antiviral agents, chemotherapeutic agents (i.e., anticancer agents), anti-rejection agents, analgesics and analgesic combinations, anti-inflammatory agents, steroids, or any combination thereof.

### Printing Systems:

Preferably, the bioprinted tissue fibers described herein are printed using LOP™ technology as described in PCT/CA2014/050556, PCT/CA2018/050315, and U.S. Ser. No. 62/733,548; the disclosures of which are expressly incorporated herein by reference. As detailed therein, the LOP™ bioprinting system enables multi-material switching, and thus the composition of the vessel wall (cell type and biomaterial composition) can be modified along the length of the channel while continuously printing.

In an exemplary embodiment, the printing system comprises a print head comprising a dispensing channel, wherein one or more material channels and a core channel converge at the proximal end of the dispensing channel. The subject print heads may be configured to dispense buffer solution and/or sheath fluid simultaneous with one or more cross-linkable materials. In some embodiments, a print head is configured to maintain a constant mass flow rate through the dispensing channel. In this manner, the subject print heads are configured to facilitate a smooth and continuous flow of one or more input materials (or a mixture of one or more input materials) and a buffer solution and/or sheath fluid through the dispensing channel. In use of the subject print heads, an input material flowing through the dispensing channel can be cross-linked from the inside, by a fluid flowing through the core channel and/or from the outside, by sheath fluid flowing through a downstream sheath fluid channel, as described more particularly in WO2020/056517, the disclosure of which is expressly incorporated herein by reference.

In a preferred embodiment, a print head comprises a dispensing channel with a proximal end and a distal end; a dispensing orifice located at the distal end of the dispensing channel; one or more shell channels that converge sequentially with the dispensing channel at the distal end of the dispensing channel, wherein each shell channel has a convergence angle of between about 20 and 90 degrees, a core channel that converges with the dispensing channel at the proximal end of the dispensing channel, wherein the core channel has a convergence angle of 0 degrees; and, optionally a sheath flow channel that diverges into two sheath flow sub-channels, wherein the sheath flow sub-channels converge with the dispensing channel at a sheath fluid intersection and have a convergence angle of between about 30 and 60 degrees, more preferably between about 40 and 50 degrees, most preferably about 45 degrees.

Additional aspects include printing systems and associated components that are configured to work in conjunction with the subject print heads to prepare the subject fibers. In some embodiments, a printing system comprises a single print head, as described herein. In some embodiments, a printing system comprises a plurality of print heads, such as 2, 3, 4, 5, 6, 7, 8, 9 or 10 individual print heads, as described herein. In some embodiments, a print head is fluidically isolated from a printing system, such that all fluids involved with the printing process remain isolated within the print head, and only make contact with a receiving surface of the printing system (described below) during the printing process. In some embodiments, a print head is configured to be operably coupled to a printing system without bringing the fluids involved with the printing process into contact with the components of the printing system. In some embodiments, one or more print heads can be removed and/or added to a printing system before, during and/or after a printing process. Accordingly, in some embodiments, the subject print heads are modular components of the subject printing systems.

In some embodiments, a printing system comprises a receiving surface upon which a first layer of material dispensed from a dispensing orifice of a print head is deposited. In some embodiments, a receiving surface comprises a solid material. In some embodiments, a receiving surface comprises a porous material. For example, in some embodiments, the porosity of the porous material is sufficient to allow passage of a fluid there through. In some embodiments, a receiving surface is substantially planar, thereby providing a flat surface upon which a first layer of dispensed material can be deposited. In some embodiments, a receiving surface has a topography that corresponds to a three dimensional structure to be printed, thereby facilitating printing of a three dimensional structure having a non-planar first layer.

In some embodiments, a receiving surface comprises a vacuum component that is configured to apply suction from one or more vacuum sources to the receiving surface. In some embodiments, a receiving surface comprises one or more vacuum channels that are configured to apply suction to the receiving surface. In some embodiments, a receiving surface comprising a vacuum component is configured to aspirate an excess fluid from the receiving surface before, during and/or after a printing process is carried out.

In some embodiments, a printing system achieves a particular geometry by moving a print head relative to a printer stage or receiving surface adapted to receive printed materials. In other embodiments, a printing system achieves a particular geometry by moving a printer stage or receiving surface relative to a print head. In certain embodiments, at least a portion of a printing system is maintained in a sterile environment (e.g., within a biosafety cabinet (BSC)). In some embodiments, a printing system is configured to fit entirely within a sterile environment.

] In some embodiments, a printing system comprises a 3D motorized stage comprising three arms for positioning a print head and a dispensing orifice in three dimensional space above a print bed, which comprises a surface for receiving a printed material. In one embodiment, the 3D motorized stage (i.e., the positioning unit) can be controlled to position a vertical arm, which extends along the z-axis of the 3D motorized stage such that the print head orifice is directed downward. A first horizontal arm, which extends along the x-axis of the motorized stage is secured to an immobile base platform. A second horizontal arm, which extends along the y-axis of the motorized stage is moveably coupled to an upper surface of the first horizontal arm such that the longitudinal directions of the first and second horizontal arms are perpendicular to one another. It will be understood that the terms “vertical” and “horizontal” as used above with respect to the arms are meant to describe the manner in which the print head is moved and do not necessarily limit the physical orientation of the arms themselves.

In some embodiments, a receiving surface is positioned on top of a platform, the platform being coupled to an upper surface of the second horizontal arm. In some embodiments, the 3D motorized stage arms are driven by three corresponding motors, respectively, and controlled by a programmable control processor, such as a computer. In a preferred embodiment, a print head and a receiving surface are collectively moveable along all three primary axes of a Cartesian coordinate system by the 3D motorized stage, and movement of the stage is defined using computer software. It will be understood that the invention is not limited to only the described positioning system, and that other positioning systems are known in the art. As material is dispensed from a dispensing orifice on a print head, the positioning unit is moved in a pattern controlled by software, thereby creating a first layer of the dispensed material on the receiving surface. Additional layers of dispensed material are then stacked on top of one another such that the final 3D geometry of the dispensed layers of material is generally a replica of a 3D geometry design provided by the software. The 3D design may be created using typical 3D CAD (computer aided design) software or generated from digital images, as known in the art. Further, if the software generated geometry contains information on specific materials to be used, it is possible, according to one embodiment of the invention, to assign a specific input material type to different geometrical locations. For example, in some embodiments, a printed 3D structure can comprise two or more different input materials, wherein each input material has different properties (e.g., each input material comprises a different cell type, a different cell concentration, a different ECM composition, etc.).

Aspects of the subject printing systems include software programs that are configured to facilitate deposition of the subject input materials in a specific pattern and at specific positions in order to form a specific fiber, planar or 3D structure. In order to fabricate such structures, the subject printing systems deposit the subject input materials at precise locations (in two or three dimensions) on a receiving surface. In some embodiments, the locations at which a printing system deposits a material are defined by a user input, and are translated into computer code. In some embodiments, a computer code includes a sequence of instructions, executable in the central processing unit (CPU) of a digital processing device, written to perform a specified task. In some embodiments, printing parameters including, but not limited to, printed fiber dimensions, pump speed, movement speed of the print head positioning system, and cross-linking agent intensity or concentration are defined by user inputs and are translated into computer code. In some embodiments, printing parameters are not directly defined by user input, but are derived from other parameters and conditions by the computer code.

In some embodiments, the locations at which a printing system deposits an input material are defined by a user input and are translated into computer code. In some embodiments, the devices, systems, and methods disclosed herein further comprise non-transitory computer readable storage media or storage media encoded with computer readable program code. In some embodiments, a computer readable storage medium is a tangible component of a digital processing device such as a bioprinter (or a component thereof) or a computer connected to a bioprinter (or a component thereof). In some embodiments, a computer readable storage medium is optionally removable from a digital processing device. In some embodiments, a computer readable storage medium includes, by way of non-limiting example, a CD-ROM, DVD, flash memory device, solid state memory, magnetic disk drive, magnetic tape drive, optical disk drive, cloud computing system and/or service, and the like. In some cases, the program and instructions are permanently, substantially permanently, semi-permanently, or non-transitorily encoded on a storage medium.

In some embodiments, the devices, systems, and methods described herein comprise software, server, and database modules. In some embodiments, a “computer module” is a software component (including a section of code) that interacts with a larger computer system. In some embodiments, a software module (or program module) comes in the form of one or more files and typically handles a specific task within a larger software system.

] In some embodiments, a module is included in one or more software systems. In some embodiments, a module is integrated with one or more other modules into one or more software systems. A computer module is optionally a stand-alone section of code or, optionally, code that is not separately identifiable. In some embodiments, the modules are in a single application. In other embodiments, the modules are in a plurality of applications. In some embodiments, the modules are hosted on one machine. In some embodiments, the modules are hosted on a plurality of machines. In some embodiments, the modules are hosted on a plurality of machines in one location. In some embodiments, the modules are hosted a plurality of machines in more than one location. Computer modules in accordance with embodiments of the invention allow an end user to use a computer to perform the one or more aspects of the methods described herein.

In some embodiments, a computer module comprises a graphical user interface (GUI). As used herein, “graphic user interface” means a user environment that uses pictorial as well as textual representations of the input and output of applications and the hierarchical or other data structure in which information is stored. In some embodiments, a computer module comprises a display screen. In further embodiments, a computer module presents, via a display screen, a two-dimensional GUI. In some embodiments, a computer module presents, via a display screen, a three-dimensional GUI such as a virtual reality environment. In some embodiments, the display screen is a touchscreen and presents an interactive GUI.

Aspects also include one or more quality control components that are configured to monitor and/or regulate one or more parameters of the subject printing systems in order to ensure that one or more printed fibers have suitable properties. For example, in some embodiments, if a deposition process proceeds too quickly, a printed fiber structure can begin to form a coiled structure within the dispensing channel or outside the dispensing channel after it has been dispensed. In some embodiments, a quality control component comprises a camera that is configured to monitor the deposition process by collecting one or more images of a printed fiber structure, and to determine whether the printed fiber structure has formed a coiled structure. In some embodiments, a quality control component is configured to modulate one or more parameters of a deposition process (e.g., to reduce pressure and/or to reduce deposition speed) so as to diminish or avoid formation of a coiled structure by the printed fiber structure.

Aspects further include one or more fluid reservoirs that are configured to store a fluid and deliver the fluid to the printing system (e.g., the print head) through one or more fluid channels, which provide fluid communication between the printing system and the reservoirs. In some embodiments, a printing system comprises one or more fluid reservoirs that are in fluid communication with a fluid channel. In some embodiments, a fluid reservoir is connected to an input orifice of a fluid channel. In some embodiments, a fluid reservoir is configured to hold a volume of fluid that ranges from about 100 μL up to about 1 L, such as about 1, 5, 10, 15, 20, 25, 30, 35, 40, 45, 50, 55, 60, 65, 70, 75, 80, 85, 90, 95 or 100 mL, or such as about 150, 200, 250, 300, 350, 400, 450, 500, 550, 600, 650, 700, 750, 800, 850, 900 or 950 mL.

In some embodiments, a printing system comprises a pressure control unit, which is fluidly coupled to the one or more reservoirs. The pressure control unit is configured to provide a force to move one or more fluids through the printing system. In some embodiments, a pressure control unit supplies pneumatic pressure to one or more fluids via one or more connecting tubes. The pressure applied forces a fluid out of a reservoir and into the print head via respective fluid channels. In some embodiments, alternative means can be used to move a fluid through a channel. For example, a series of electronically controlled syringe pumps could be used to provide force for moving a fluid through a print head.

In some embodiments, a printing system comprises a light module (as described above) for optionally exposing a photo cross-linkable input material to light in order to cross-link the material. Light modules in accordance with embodiments of the invention can be integrated into a print head, or can be a component of a printing system.

### Additional Fluids:

Aspects of the invention include one or more buffer solutions. Buffer solutions in accordance with embodiments of the invention are miscible with an input material (e.g., a hydrogel) and do not cross-link the input material. In some embodiments, a buffer solution comprises an aqueous solvent. Non-limiting examples of buffer solutions include polyvinyl alcohol, water, glycerol, propylene glycol, sucrose, gelatin, or any combination thereof.

Buffer solutions in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa: s. In some embodiments, the viscosity of a buffer solution can be modulated so that it matches the viscosity of one or more input materials.

Aspects of the invention include one or more sheath fluids. Sheath fluids in accordance with embodiments of the invention are fluids that can be used, at least in part, to envelope or “sheath” an input material being dispensed from a dispensing channel. In some embodiments, a sheath fluid comprises an aqueous solvent. Non-limiting examples of sheath fluids include polyvinyl alcohol, water, glycerol, propylene glycol, sucrose, gelatin, or any combination thereof. Sheath fluids in accordance with embodiments of the invention can have a viscosity that ranges from about 1 mPa·s to about 5,000 mPa·s, such as about 10, 20, 30, 40, 50, 60, 70, 80, 90, 100, 200, 300, 400, 500, 600, 700, 800, 900, 1,000, 1,250, 1,500, 1,750, 2,000, 2,250, 2,500, 2,750, 3,000, 3,250, 3,500, 3,750, 4,000, 4,250, 4,500, or 4,750 mPa·s. In some embodiments, the viscosity of a sheath fluid can be modulated so that it matches the viscosity of one or more input materials.

In some embodiments, a sheath fluid comprises a chemical cross-linking agent. In some embodiments, a chemical cross-linking agent comprises a divalent cation. Non-limiting examples of divalent cations include Cd²⁺, Ba²⁺, Cu²⁺, Ca²⁺, Ni²⁺, Co²⁺, or Mn²⁺. In a preferred embodiment, Ca²⁺ is used as the divalent cation. In some embodiments, the concentration of a divalent cation in the sheath fluid ranges from about 80 mM to about 140 mM, such as about 90, 100, 110, 120 or 130 mM.

### Methods of Making:

Aspects of the invention include methods of printing a linear fiber structure, a planar structure comprising one or more fiber structures, or a three-dimensional (3D) structure comprising two or more layers of planar structures. In some embodiments, a method first comprises providing a design for a planar or 3D structure to be printed. The design can be created using commercially available CAD software. In some embodiments, the design comprises information regarding specific materials (e.g., for heterogeneous structures comprising multiple materials) to be assigned to specific locations in the structure(s) to be printed.

Aspects of the methods comprise providing one or more input materials to be dispensed by the print head. In some embodiments, one or more cell types are compatible with, and optionally dispensed within, an input material. In some embodiments, a sheath fluid serves as a lubricating agent for lubricating movement of an input material within the print head. In some embodiments, a sheath fluid comprises a cross-linking agent for solidifying at least a portion of the hydrogel before or while it is dispensed from the print head. In some embodiments, a cross-linking agent may be included in an input material, for example an input material corresponding to a core of a fiber of the present disclosure.

Aspects of the methods comprise communicating the design to the 3D printer. In some embodiments, communication can be achieved, for example, by a programmable control processor. In some embodiments, the methods comprise controlling relative positioning of the print head and the receiving surface in three dimensional space, and simultaneously dispensing from the print head the input material, and, in some embodiments, a sheath fluid, alone or in combination. In some embodiments, the materials dispensed from the print ahead are dispensed coaxially, such that the sheath fluid envelopes the input material. Such coaxial arrangement allows a cross-linking agent in the sheath fluid to solidify the input material, thereby resulting in a solidified fiber structure, which is dispensed from the printer head.

In some embodiments, a method comprises depositing a first layer of the dispensed fiber structure on a receiving surface, the first layer comprising an arrangement of the fiber structure specified by the design, and iteratively repeating the depositing step, depositing subsequent fiber structures onto the first and subsequent layers, thereby depositing layer upon layer of dispensed fiber structures in a geometric arrangement specified by the design to produce a 3D structure.

In some embodiments, a plurality of input materials, for example multiple hydrogels, at least some of which comprise one or more cell types, are deposited in a controlled sequence, thereby allowing a controlled arrangement of input materials and cell types to be deposited in a geometric arrangement specified by the design.

Aspects of the invention include methods of making a 3D structure comprising one or more input materials. The 3D structures find use in repairing and/or replacing at least a portion of a damaged or diseased tissue in a subject.

As described above, any suitable divalent cation can be used in conjunction with the subject methods to solidify a chemically cross-linkable input material, including, but not limited to, Cd²⁺, Ba²⁺, Cu²⁺, Ca²⁺, Ni²⁺, Co²⁺, or Mn²⁺. In a preferred embodiment, Ca²⁺ is used as the divalent cation. In one preferred embodiment, a chemically cross-linkable input material is contacted with a solution comprising Ca²⁺ to form a solidified fiber structure. In some embodiments, the concentration of Ca²⁺ in the sheath solution ranges from about 80 mM to about 140 mM, such as about 90, 100, 110, 120 or 130 mM.

In certain embodiments, an input material is solidified in less than about 5 seconds, such as less than about 4 seconds, less than about 3 seconds, less than about 2 seconds, or less than about 1 second.

Aspects of the invention include methods of depositing one or more input materials in a patterned manner, using software tools, to form layers of solidified structures that are formed into a multi-layered 3D tissue structure. In some embodiments, a multi-layered 3D tissue structure comprises a plurality of mammalian cells. Advantageously, by modulating the components (e.g., the mammalian cell type, cell density, matrix components, active agents) of the subject input materials, a multi-layered 3D tissue structure can be created using the subject methods, wherein the multi-layered 3D tissue structure has a precisely controlled composition at any particular location in three dimensional space. As such, the subject methods facilitate production of complex three dimensional tissue structures.

### Methods of Using:

Aspects of the invention include methods of using a bioprinted tissue fiber for evaluating immune cell infiltration of tumor cells. In some embodiments, the bioprinted tissue fiber for evaluating immune cell infiltration of tumor cells comprises a first region that comprises a plurality of tumor cells embedded in a first cross-linkable biocompatible material; a second region that comprises a plurality of stromal cells embedded in a second cross-linkable biocompatible material; and a third region that comprises at least one immune cell population of interest embedded in a third cross-linkable biocompatible material.

The methods of using involve providing an in vitro model of tumor cell microenvironment, comprising at least one bioprinted tissue fiber as herein described. In some embodiments, the methods comprise providing a customizable theranostic device for evaluating a cancer immunotherapy in a patient in need thereof, comprising at least one bioprinted tissue fiber as herein described. In some embodiments, in the theranostic device at least one of, at least two of, or each of the plurality of tumor cells, the plurality of CAF, and/or the at least one immune cell population of interest are patient-specific.

In some embodiments, the method of using comprises evaluating the compatibility and/or efficacy of a candidate agent, comprising contacting and/or printing as herein described with the candidate agent and detecting immune cell infiltration into the core of the fiber.

In some embodiments, the method of using comprises screening a candidate agent for anti-cancer activity, the method comprising: contacting a bioprinted tissue fiber as herein described with the candidate agent; and measuring an anti-cancer response in at least one cell type in the bioprinted tissue fiber; wherein an increase in the anti-cancer response in the presence of the candidate agent indicates that the candidate agent has anti-cancer activity.

The methods involve drug assay using the bioprinted tissue fiber as herein described. In embodiments, cell culture media containing compounds to test and assay media were provided to a single or multiple plate. The bioprinted tissue fiber is transferred within culture media to the plate containing assay media and incubated with appropriate condition. In embodiments, the incubation occurs in 5% CO₂ at 37 ° C for up to 6 days and media is changed every 2-3 days.

In some embodiments, the bioprinted tissue fiber is fixed/crosslinked. After incubation, the bioprinted tissue fiber is transferred to fixation/crosslinking solution and subsequently incubated/washed for confocal imaging. The confocal imaging is acquired with three-dimensional imaging for z-stacks under laser at different time-point. Surfaces segmentation was performed for tumor organoids and region of interest (ROI) for the core of the bioprinted fiber was identified. In embodiments, the surfaces/spots statistics were exported to evaluate the efficacy of drugs being assayed, on the tissue growth (number of objects, area and volume), CAFs contraction and spreading (ellipticity and sphericity), T cells infiltration (position, shortest distance (direct cell-to-cell contact threshold of <10 μm distance)), and PDXOs apoptosis (shortest distance (colocalization threshold of <1 μm distance)).

All patents and patent publications referred to herein are hereby incorporated by reference in their entirety.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

### Examples

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Novel Printheads to Enable Accurate Printing of Tumor Cells, CAFs and TILs and Recreate the Spatial Distribution of ‘Hot’ and ‘Cold’ Tumors

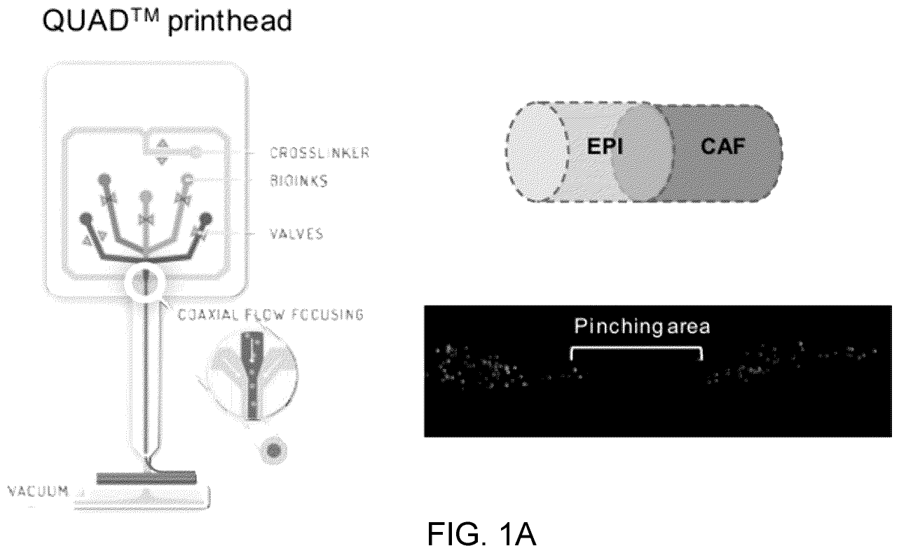

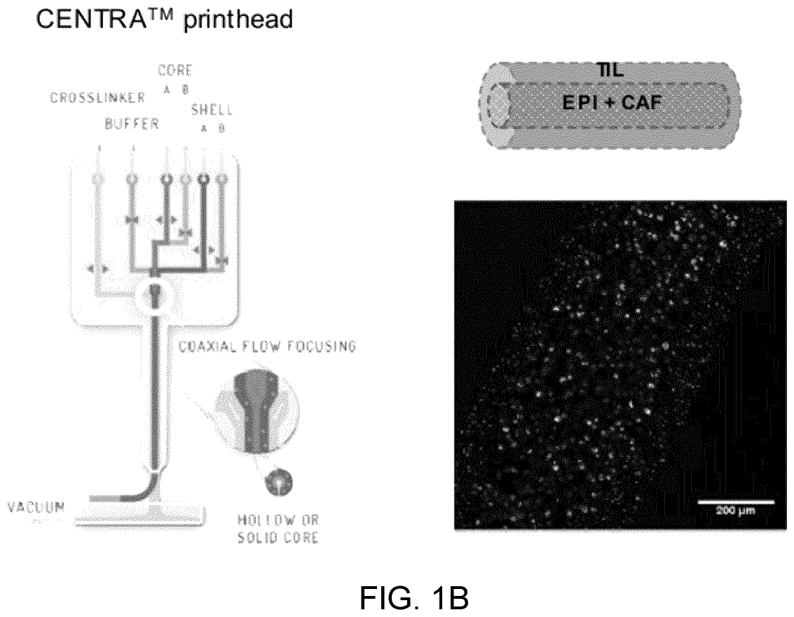

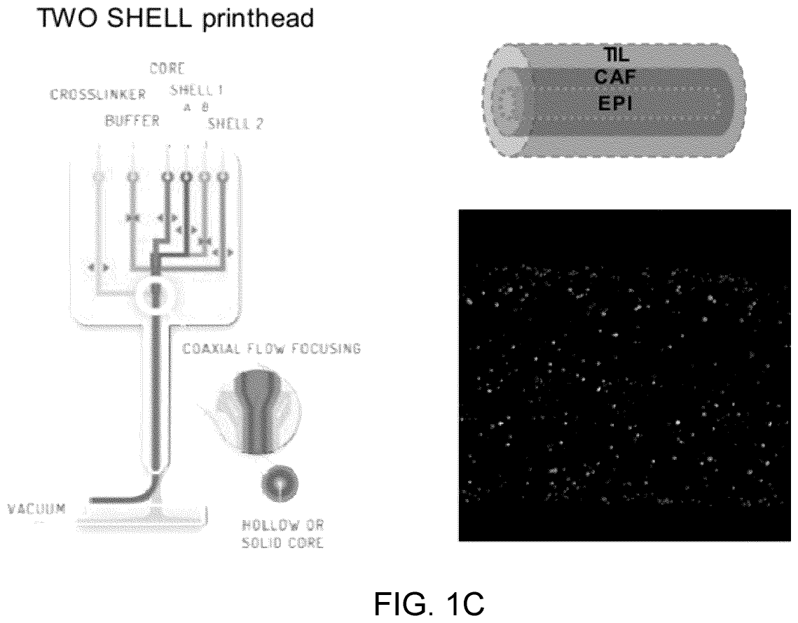

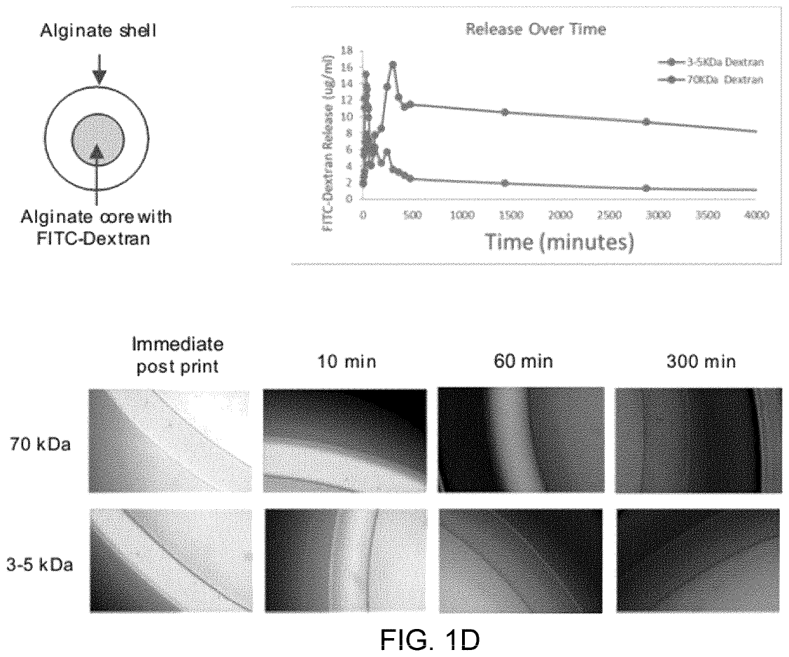

Rapid material switching procedures were developed to enable patterning in defined regions within a coded fiber. The first design consisted of bioprinting sequential compartments along the length of a fiber using the QUAD™ printhead (FIG. 1A). Printing of three different cell types within specific regions of the fiber required switching between materials by near-instantaneous flow. However, bioink required an initial push to flow due to gelation in tube. Also, pinching occurred when switching between materials due to valve opening. Consequently, the design was not optimal due to interspaced pinched region without cells. To use TIL infiltration as a key readout for the 3D-TNBC tissue assay, a core-shell fiber printing technology was developed with the CENTRA™ printhead to allow monitoring of TILs migration from the shell into the core, containing tumor cells and CAFs. the tumor cells and CAFs to be printed in a core and TILs adjacent shell layer to model tumor-TIL boundaries (FIG. 1B). In order to better address the effect of the CAF-induced fibrotic barrier on TILs infiltration, a TWO SHELL printhead prototype was developed to enable printing of a multi-layer core-two-shell fiber, allowing the three cell components to be patterned in a core and adjacent shell layer to model tumor-CAF-TIL boundaries (FIG. 1C).

Biomaterial Composition that Supports Printing of Viable Tumor Cells, CAFs and TILs Separately

As a first step towards demonstrating appropriate biological response of the printed tumor tissues to antibody-based checkpoint inhibitor immunotherapies, we demonstrated the alginate biomaterials used for bioprinting are permeable to biomolecules of various sizes equivalent to antibodies (IgG) (FIG. 1D). Cell-free core-shell fibers were printed using prototype CENTRA™ printhead, with the core material loaded with FITC-dextran of defined molecular weights and hydrodynamic radii. The shell of the fibers was a 2-300 micron outer layer of alginate-based material without FITC-dextran. Different fibers were loaded with different variants of FITC-dextran to model small molecule diffusion (3-5 kDa) and diffusion of larger antibody-based molecules (70 kDa FITC-dextran with a similar hydrodynamic radius to IgG). Real-time fluorescent imaging of the printed fibers and corresponding sampling of conditioned media analyzed on a fluorescent plate reader were performed to assess FITC-dextran diffusion qualitatively and quantitatively through the fiber respectively. Both assays demonstrate the 3-5 kDa molecule diffuses rapidly through the fiber material, measurable in the conditioned medium within 10 minutes, reaching a plateau after around 60 minutes. Diffusion of the larger 70 kDa FITC-dextran molecule was slower, but the majority still diffused through the fiber within 2-3 hours. This study demonstrates that molecules of a similar hydrodynamic radius to IgG-antibodies can freely diffuse through the bioprinted alginate-based materials, although the speed of diffusion is slightly delayed for larger molecules. Confocal imaging analysis revealed that tumor cells and CAF embedded in AGC-10TM biomaterial were encapsulated by the alginate gel which impaired CAF elongation/contraction and interaction with cancer cells. We tested the addition of 10% and 25% Matrigel in the AGC-10™ bioink and observed that 25% Matrigel enabled fibroblasts remodelling and cancer cell spreading (FIG. 1E).

### Staining Assays for Assessment of Tumor Cell Growth/Death and TIL Migration/Activation Using Live Cell Fluorescent Imaging

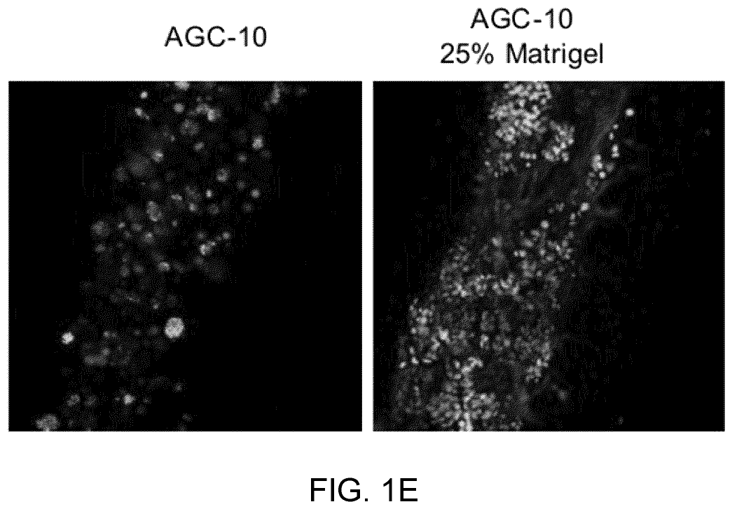

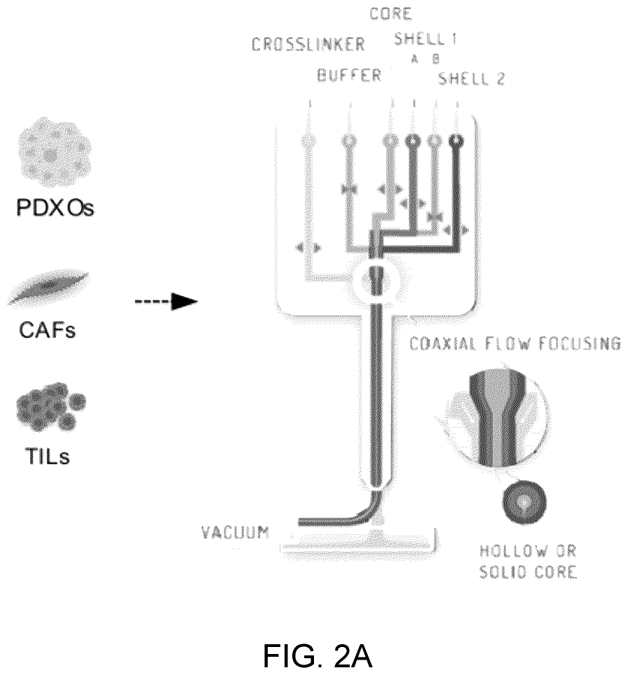

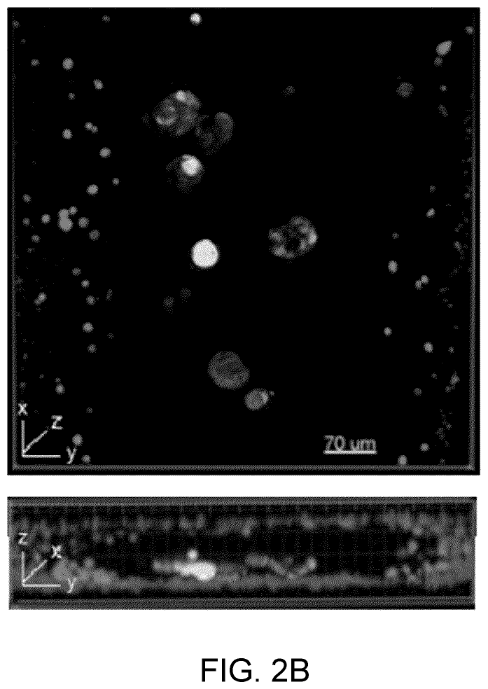

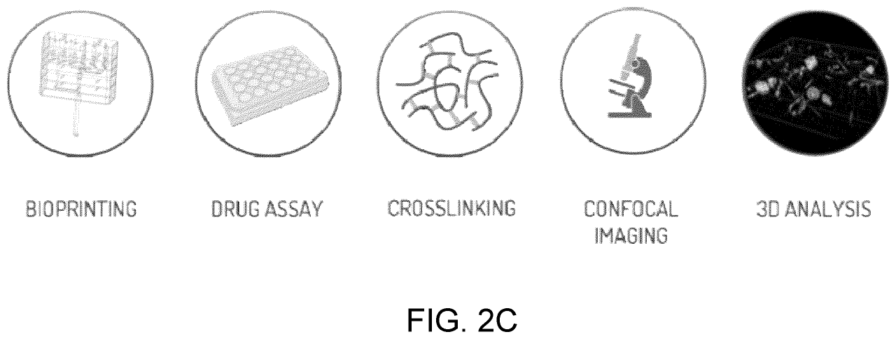

We have genetically engineered tumor organoids or cells to express green fluorescent protein (GFP) and CAF to express red fluorescent protein (tdTomato). Alternatively, tumor organoids have been loaded with Cell Tracker Green CMFDA dye (Molecular Probes) before bioprinting. TILs were loaded with Cell Tracker Deep Red dye before bioprinting (FIG. 2A-B). Tumor cell death was monitored using fluorescent caspase- 3/7 substrates (NucView® 405 Caspase-3 substrate, Biotium). We successfully developed a 5-steps 3D-TNBC tissue assay using patient-derived TME components that enable phenotypic drug screening (Deliverables 3 and 4) (FIG. 2C): Step 1) Bioprinting of fluorescent-labelled patient-derived PDXOS, CAFs and TILs using TWO SHELL prototype printhead; Step 2) 3D-TNBC tissue culture and drug assay for up to 6 days; Step 3) Crosslinking and fixation of 3D-TNBC tissue at endpoint; Step 4) Confocal z-stacks imaging; Step 5) 3D image analysis to quantify 3D-TNBC tissue growth, ECM remodelling, tumor organoids apoptosis and TILs infiltration.

### Quantification of Tumor Cell Death and TIL Migration/Activation on the Opera Phenix (or Similar) High Content Imaging System

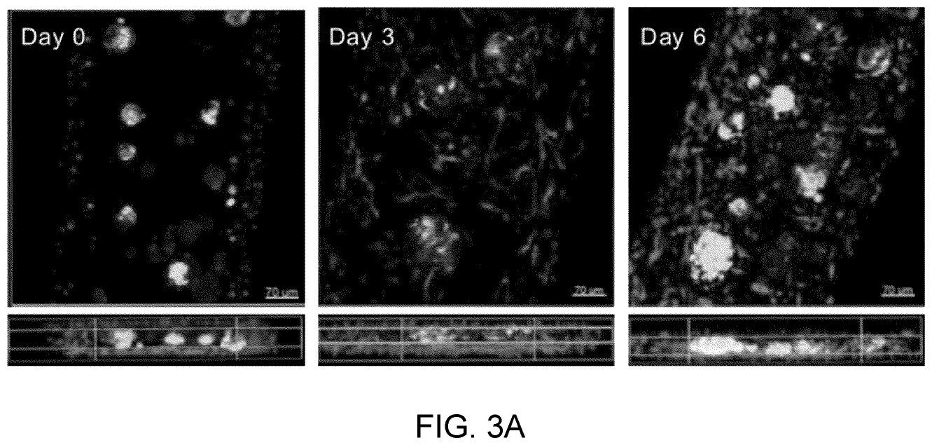

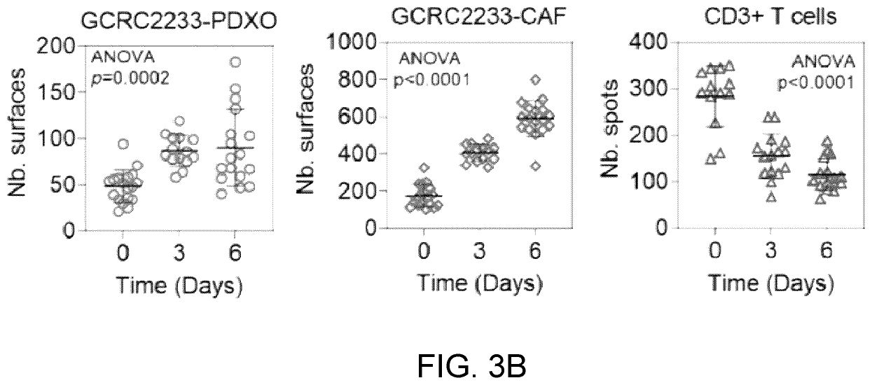

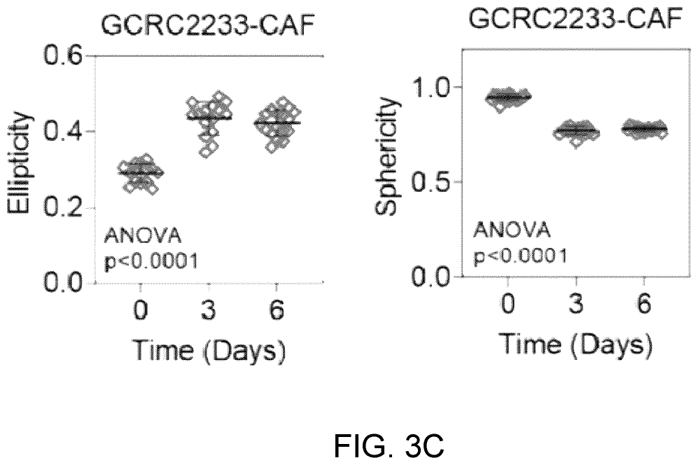

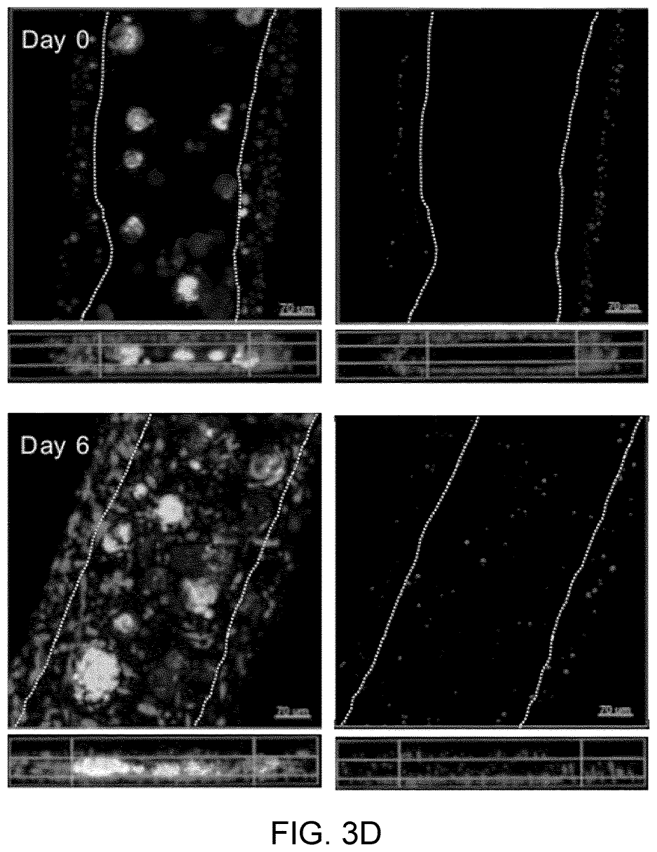

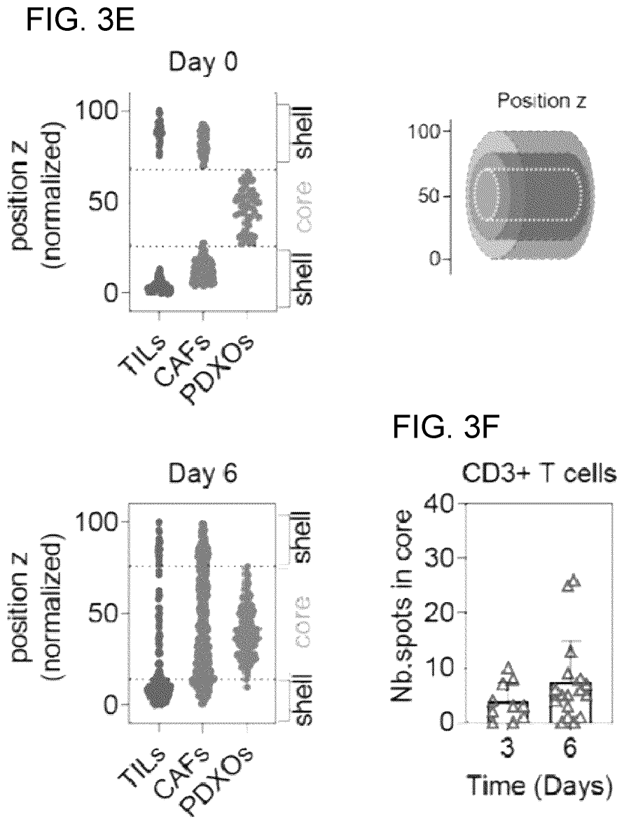

3D image analysis of confocal z-stacks was performed using Imaris software to enable image-based quantification of TME components growth, ECM remodeling, TILs infiltration and PDXOs apoptosis. We have demonstrated that the 3D-TNBC tissue sustains growth of tumor organoids and CAF during 6 days in culture based on number of surfaces detected from green and red fluorescence channels (FIGS. 3A and B). T cells number of spots detected decreased over time, which reflects the activation and exhaustion states of T cells at the time of printing. Measurements of ellipticity and sphericity of CAF surfaces confirm that 3D-TNBC tissue allows ECM remodelling necessary for CAF activation (FIG. 3C). We observed that 3D-TNBC tissue allows T cells infiltration from the outer shell into the tumor core of the bioprinted fiber over time (FIG. 3D). T cells infiltration into the tumor core of the fiber was quantified by monitoring position of surfaces and spots in the z plane, normalized on the highest and lowest position measured from the outer shell of the fiber (FIG. 3E), and by quantifying number of spots detected into the core delineated from PDXOs position xyz (FIG. 3F).

Bioprinted Tumor Models have an Appropriate Biological Response to Clinically Proven Therapy

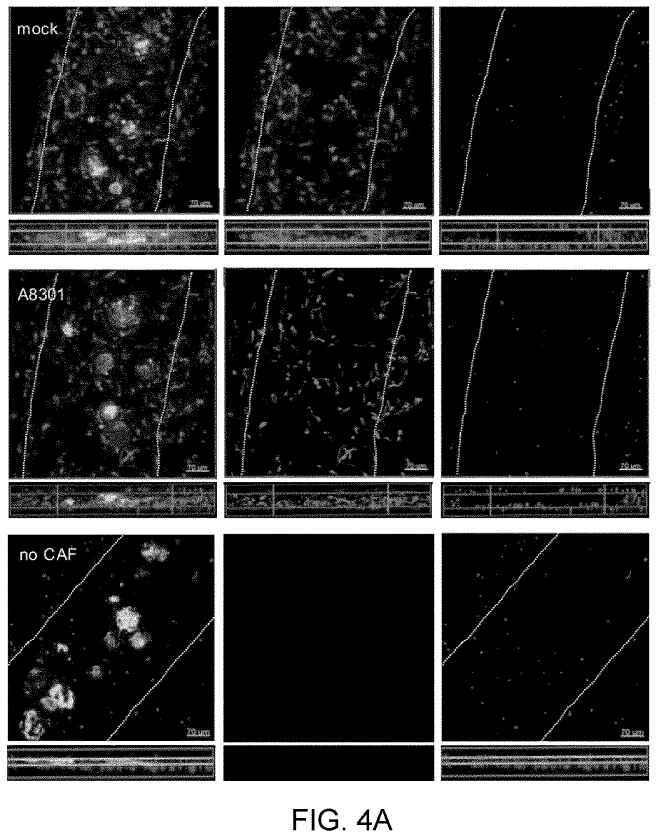

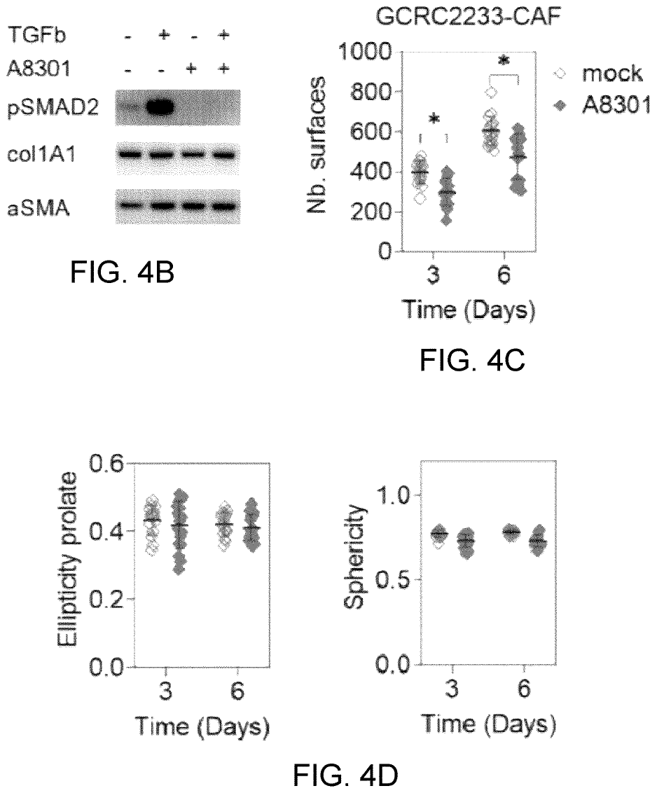

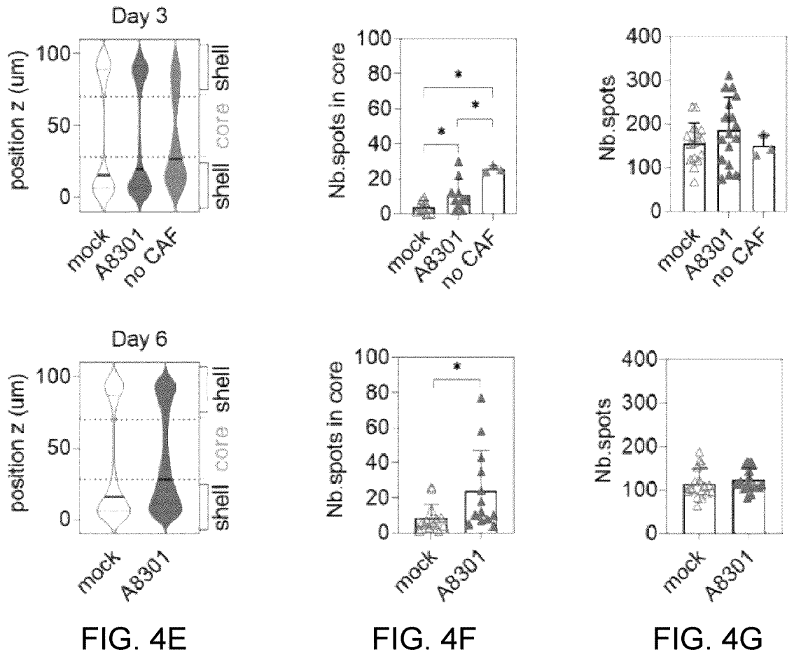

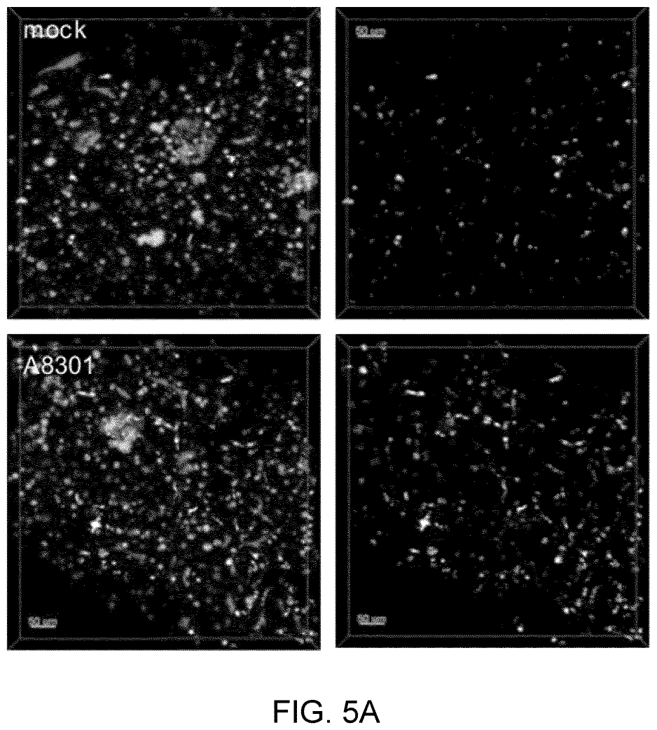

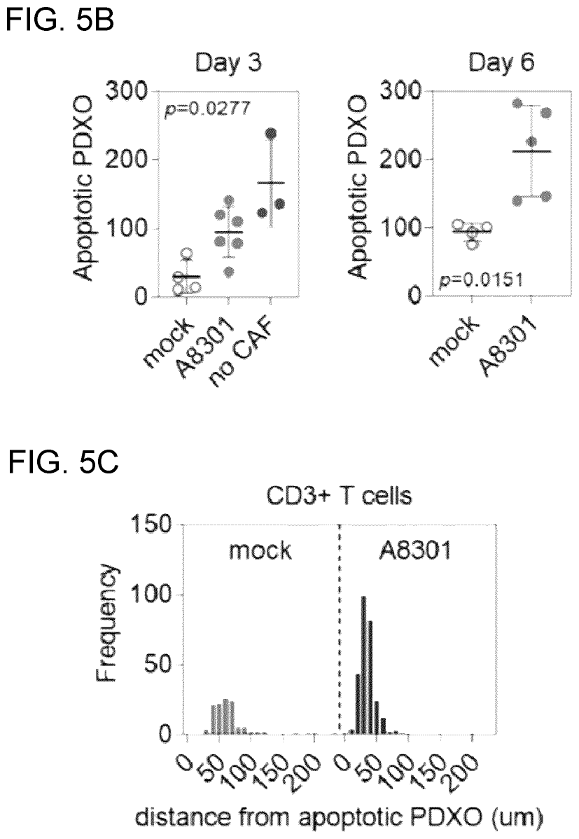

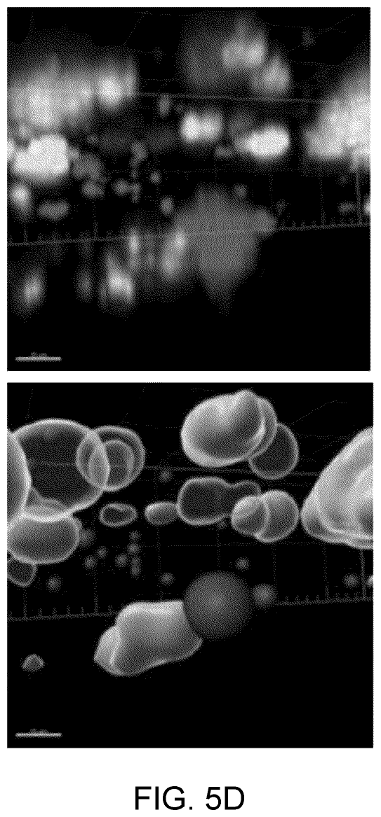

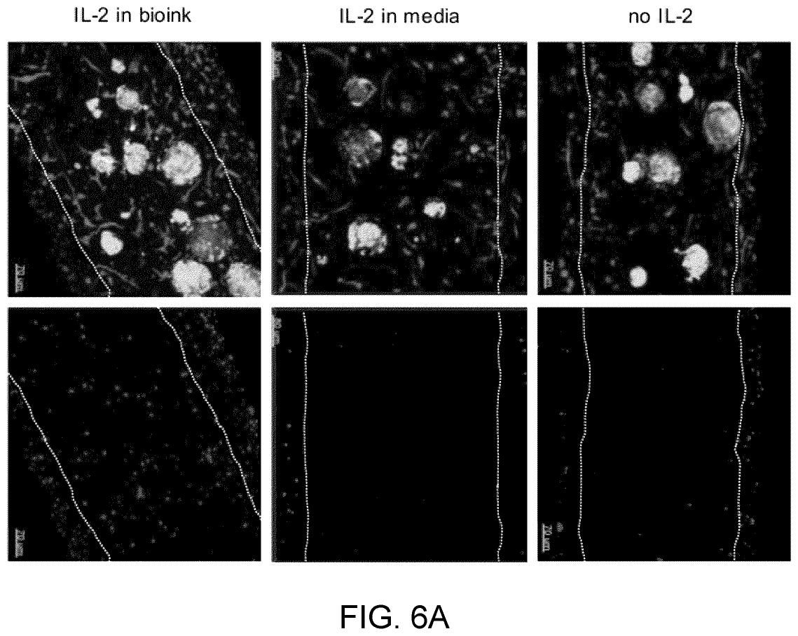

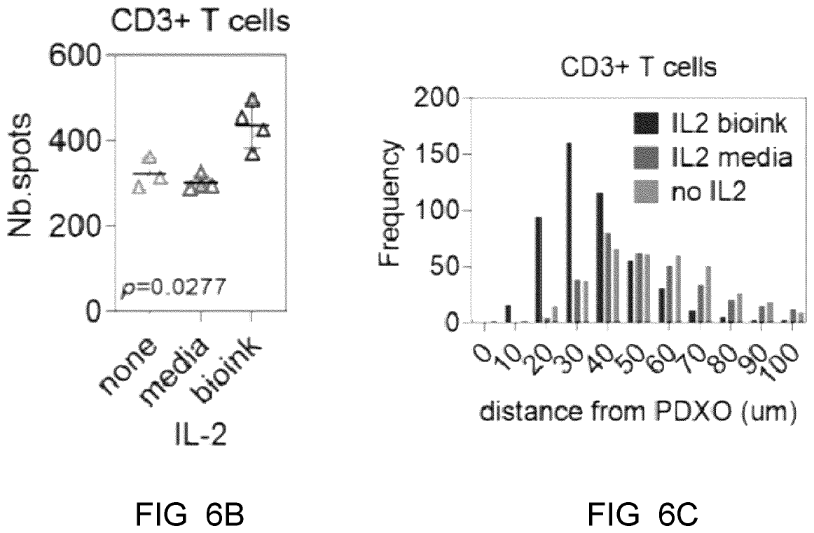

As a proof-of-concept, pharmacological response was tested using drug with known clinical effects. TGF-beta inhibition has been reported to improve T cell infiltration into the tumor and synergize with immune checkpoint therapy (5, 6). 3D-TNBC tissue was culture for 6 days in presence of mock treatment (DMSO 0.1%) or TGF-beta small molecule inhibitor A8301 (5 μM). As a positive control, we also printed a fiber without CAFs in the inner shell biomaterial (no CAF) (FIG. 4A). We demonstrated that A8301 inhibitor effectively blocked TGF-beta downstream signaling target phospho-SMAD2 in GCRC2233-CAF (FIG. 4B), significantly decreased number of CAFs surfaces (FIG. 4C) but had no effect on CAFs elongation/contraction based on ellipticity and sphericity socres (FIG. 4D). T cells infiltration into the tumor core of the fiber was significantly increased under TGF-beta inhibitor exposure and in absence of CAFs in the 3D-TNBC tissue, as monitored by the normalized position of spots in the z plane (FIG. 4E) and by the number of spots detected into the core (FIG. 4F). Total number of spots detected in the 3D-TNBC tissue was not affected by TGF-beta inhibition (FIG. 4G). Tumor cell death was assessed using fluorescent caspase- 3/7 substrates (NucView® 405 Caspase-3 substrate, Biotium) by quantifying number of PDXOs surfaces that colocalize (<1 μm distance) with Nuc View surfaces (FIGS. 5A and B). Our results showed that TGF-beta inhibition significantly increased number of apoptotic PDXOs over time (FIG. 5B). T cell killing of the tumor PDXOs was evaluated by the frequency of T cells in shortest distance to apoptotic PDXOs, which was increased after 6 days of A8301 exposure compared to mock treatment (FIGS. 5C and D). Interleukin-2 (IL-2) acts as a differentiation factor that promote the expansion of CD8 T cells and induce the expression of co-inhibitory receptors. We tested if the addition of IL-2 in the media or the bioink could improve TILs survival and infiltration in the 3D-TNBC tissue after 3 days in culture (FIG. 6A). Our data showed that adding 150 IU/mL IL-2 in the bioink along with T cells significantly increased TILs number after 3 days in culture as determined by the number of spots detected (FIG. 6B). Presence of IL-2 in the bioink also promoted TILs infiltration into the core, indicated by an increased frequency of T cells that are in a short distance from PDXOs (FIG. 6C).

3D-TNBC tissue assay

### 1. Description

### 1.1. Purpose

The following protocol is optimized to produce the 3D-TNBC tissue assay using fluorescent labeled patient-derived cells and to measure tissue growth, ECM remodelling, tumor cell apoptosis and immune cell infiltration. This assay enables phenotypic screening in a physiological human tissue to efficiently identify novel anti-cancer molecules and immuno-oncology therapeutics.

### 1.2. Material

Patient-derived cells (tumor organoids, cancer-associated fibroblasts and T cells)
CellTracker™ Green CMFDA Dye (Invitrogen #C7025)
CellTracker™ Red CMTPX Dye (Invitrogen #C34552)
CellTracker™ Deep Red Dye (Invitrogen #C34565)
NucView® 405 Caspase-3 substrate (Biotium #PI-10407)
DMSO
PBS
DMEM
Culture media
AGC-10™ bioink (Aspect Biosystems)
Matrigel Growth Factor Reduced (Corning #356231)
Sterile water
60 mm dish
6-well plate
12-well plate
15 mL tube
Aspect RX1 bioprinter, accessories and software
Aspect TWO SHELL prototype TP153 printhead
Aspect Studio design file TP153 AGC-10 Multi-Shell and Core.apj
Transfer pipettes (Fisher Scientific #13-711-7M)
Paraformaldehyde 4%
Calcium chloride 1M (Sigma #21115)
Microscope slides (Fisher Scientific #12-544-2)
Rectangle cover glasses (Fisher Scientific #12-545E).
Shandon Immu-Mount (Fisher Scientific #9990402)
Confocal microscope
Imaris software (Oxford Instruments Group)

### 1.3. Solution Recipes

### 1.3.1. Culture Media

| Final [ ] | Reagent | Stock [ ] | Cie | # catalog | 500 mL |
|---|---|---|---|---|---|
| 1X | DMEM | 1X | Gibco | 11995073 | 450 mL |
| 10% | FBS | 1X | Gibco | | 50 mL |
| 50 ug/mL | Gentamicin | 10 mg/mL | Gibco | 15710072 | 2.5 mL |

### 1.3.2. Fixation/Crosslinking Solution

| | | Stock | | # | |
|---|---|---|---|---|---|
| Final [ ] | Reagent | [ ] | Cie | catalog | 20 mL |
| 4% | Paraformaldehyde | 16% | EMS | 15710 | 5 mL |
| 100 mM | Calcium chloride | 1M | Sigma | 21115 | 2 mL |
| n/a | Sterile water | n/a | n/a | n/a | 13 mL |

### 1.3.3. Bioink

| Final [ ] | Reagent | Cie | # catalog | 5 mL |
|---|---|---|---|---|
| 75% | AGC-10 ™ | Aspect Biosystems | | 3.75 mL |
| 25% | Matrigel | Corning | 356231 | 1.25 mL |

### 2. Protocol

### 2.1. Fluorescent Labeling of Patient-Derived Cells

### 2.1.1. CellTracker™ Green CMFDA Staining of Tumor Organoids

1. Prepare 10 mM stock solution in DMSO.
2. Dilute the stock solution to a final working concentration of 5 μM in DMEM.
3. Warm the working solution to 37 °C.
4. Resuspend tumor organoids gently in pre-warmed working solution.
5. Incubate 15-45 minutes at 37 °C.
6. Centrifuge tumor organoids 1200 rpm 4 min 4 ° C and discard working solution.
7. Wash tumor organoids twice in PBS.
8. Keep in PBS on ice until step 2.2.

### 2.1.2. CellTracker™ Red CMTPX Staining of Fibroblasts

9. Prepare 10 mM stock solution in DMSO.
10. Dilute the stock solution to a final working concentration of 5 μM in DMEM.
11. Warm the working solution to 37 °C.
12. Resuspend fibroblasts gently in pre-warmed working solution.
13. Incubate 15-45 minutes at 37 °C.
14. Centrifuge fibroblasts 1200 rpm 4 min 4 ° C and discard working solution.
15. Wash fibroblasts twice in PBS.
16. Keep in PBS on ice until step 2.2.

### 2.1.3. CellTracker™ Deep Red Staining of T Cells

17. Prepare 1 mM stock solution in DMSO.
18. Dilute the stock solution to a final working concentration of 1 μM in DMEM.
19. Warm the working solution to 37 °C.
20. Resuspend T cells gently in pre-warmed working solution.
21. Incubate 15-45 minutes at 37 °C.
22. Centrifuge T cells 1200 rpm 4 min 4 ° C and discard working solution.
23. Wash T cells twice in PBS.
24. Keep in PBS on ice until step 2.2.

### 2.2. Preparation of Biomaterials

25. Prepare one 15 mL conical tube for each biomaterial.
26. Resuspend 8×10\hat{ }5 fluorescent labeled tumor organoids (~50 μm diameter) in 1 mL bioink (see section 1.3.3 for solution recipe).
27. Resuspend 2×10\hat{ }6 fluorescent labeled fibroblasts in 1 mL bioink.
28. Resuspend 1×10\hat{ }7 fluorescent labeled T cells in 1 mL bioink.
29. Keep on ice.

### 2.3. Bioprinting

30. Turn on the printer by pressing the power switch at the rear
31. Open the Aspect Studio software
32. Load design file TP153 AGC-10 Multi-Shell and Core.apj
33. Press Connect in the Printer Controller tab and wait for the system to initialize
34. Place a small petri dish containing sterile water on top of the vacuum chuck to collect 3D-TNBC tissue
35. Place a small petri dish beside the vacuum chuck to collect waste during priming
36. Attach TWO SHELL TP153 prototype printhead to the RX1 Bioprinter
37. Attach valve tubing to the printhead
38. Connect the buffer, crosslinker, and bioink material reservoirs to the RX1 Bioprinter
39. Connect the fluid tubing from the crosslinker, buffer, and bioink reservoirs to the printhead using the accompanying Connection Schematic for the printhead in use
40. Press Home to move the printhead to the default home position, use the motion controls to refine the position (0.2-0.3 mm inside the water), and press Set Home
41. Press Waste to move the printhead to the default waste position, use the motion controls to position the printhead above the petri dish, and press Set Waste
42. Switch Pressure to the ON position to turn on the pressure source
43. Set the pressure for each material to a value appropriate for priming (200 mbar is standard)
44. Prime the printhead according to the accompanying Priming Guide for the printhead in use
45. Set the material pressures:
   a. Shell-1A (fibroblasts): 80 mbar
   b. Shell-1B: 0 mbar
   c. Core (tumor organoids): 80 mbar
   d. Buffer: 100 mbar
   e. Crosslinker: 80 mbar
   f. Shell-2 (T cells): 80 mbar
46. Press Apply Pressure
47. Set the speed setting at 20 mm/see
48. Press Home to move into print position
49. Press Print to begin printing
50. Replace the small petri dish containing the 3D-TNBC tissue
51. Transfer the 3D-TNBC tissue into a 6-well plate containing 3 mL/well culture media.
52. To print again, repeat steps 46 through 49.
53. Press Zero Pressures followed by Apply Pressures and wait for the channel pressures to stabilize
54. Disconnect each fluid tube from the printhead
55. Disconnect each fluid tube from its respective material reservoir and discard
56. Carefully remove the valve tubing from the printhead by grasping the steel tip, using one hand to pull on the tip and the other to brace the printhead
57. Remove the printhead and discard
58. Close the Aspect Studio software
59. Turn off the printer by pressing the power switch at the back of the system
60. Clean the vacuum chuck and material reservoir caps.

### 2.4. Drug Assay

61. Prepare 2× cell culture media containing compounds to test (2× concentration) and 2 μM Nuc View® 405 Caspase-3 substrate (assay media).
62. Add 1 mL/well of assay media in a 12-well plate.
63. Transfer the 3D-TNBC tissue within 1 mL culture media from 6-well plate (step 51) into one well of the 12-well plate containing assay media.
64. Incubate in 5% CO₂ at 37 ° C for up to 6 days.

65. Change media every 2-3 days.

### 2.5. Fixation/Crosslinking

66. Transfer the 3D-TNBC tissue into a 6-well plate containing 3 mL/well PBS.
67. Transfer the 3D-TNBC tissue into a 6-well plate containing 3 mL/well fixation/crosslinking solution.
68. Incubate 10 min RT protected from light.
69. Wash the 3D-TNBC tissue twice in PBS.
70. Transfer the 3D-TNBC tissue on a microscopy slide.
71. Add Immu-mount media around the tissue.
72. Gently place a rectangle cover glass on top.
73. Let dry overnight at RT protected from light.

### 2.6. Confocal Imaging

74. Acquire three-dimensional imaging through a 20× dry objective, numerical aperture of 0.80 on a Zeiss LSM 800 confocal microscope equipped with Axio Observer Z1, using 488 nm laser for GFP and CMFDA, 561 nm laser for CMTPX and 633 nm laser for Deep Red.
75. Acquire z-stack images from a total z thickness of 100 μm with step size of 2 μm in at least three fields of view per conditions at every time-point.

### 2.7. 3D Image Analysis Using Imaris Software

76. Import datasets in Imaris 9.5.1 (Oxford Instruments Group)
77. Perform surfaces segmentation for tumor organoids (488 channel), CAFs (561 2 channel) and caspase 3 substrates (405 channel) with a filter below 100 μm” area to discard cell debris and with constant quality threshold.
78. Perform spots segmentation for TILs (640 channel) with constant quality threshold.
79. Identify region of interest (ROI) for the core of the bioprinted fiber (xy plane).
80. Export surfaces/spots statistics:
   a. For tissue growth: number of objects, area and volume
   b. For CAFs contraction and spreading: ellipticity and sphericity
   c. For T cells infiltration: position, shortest distance (direct cell-to-cell contact threshold of <10 μm distance)
   d. For PDXOs apoptosis: shortest distance (colocalization threshold of <1 μm distance)

1. A bioprinted tissue fiber for evaluating immune cell infiltration of tumor cells, comprising:
a first region that comprises a plurality of tumor cells embedded in a first cross-linkable biocompatible material;
a second region that comprises a plurality of stromal cells embedded in a second cross-linkable biocompatible material; and
a third region that comprises at least one immune cell population of interest embedded in a third cross-linkable biocompatible material.

2. The bioprinted tissue fiber of claim 1, wherein the first region, the second region, and the third region are positioned adjacent to one another within the fiber such that the second region forms an interface between the first and third regions.

3. The bioprinted tissue fiber of claim 2, wherein the bioprinted tissue fiber comprises a multi-layered core-shell fiber and the first region is a solid core, the second region comprises at least one annulus layer, and the third region comprises at least one external shell layer.

4. The bioprinted tissue fiber of any preceding claim, wherein the immune cell population of interest comprises one or more of T cells, B cells, NK cells, NKT cells, monocytes, macrophages, dendritic cells, CAR-T cells and/or CAR-NK cells.

5. The bioprinted tissue fiber of any one of claims 1-4, wherein the plurality of tumor cells comprise a solid tumor type.

6. The bioprinted tissue fiber of claim 5, wherein the solid tumor type is selected from the group comprising or consisting of breast cancer (triple negative or not), prostate cancer, skin cancer, retinoblastoma, kidney cancer, lung cancer, bone cancer, bowel cancer, brain tumor (glioblastoma etc), pancreatic cancer, insulinoma, thyroid cancer, neuroblastoma, ovarian cancer, and testicular cancer.

7. The bioprinted tissue fiber of any one of claims 1-6, wherein the plurality of stromal cells comprises cancer-associated fibroblasts (CAF).

8. The bioprinted tissue fiber of any one of claims 1-7, wherein at least one of the plurality of tumor cells, the plurality of CAF, and/or the immune cell population of interest are patient-specific.

9. The bioprinted tissue fiber of any one of claims 1-8, wherein the cross-linkable biocompatible material comprises between about between about 0.4% and 0.6% alginate, between about 1.1 and 1.3 mg/ml collagen; and between about 20% and 30% basement membrane preparation; optionally wherein the first, second and third cross-linkable biocompatible material are the same; preferably wherein the basement membrane preparation comprises or consists of matrigel.

10. The bioprinted tissue fiber of claim 9, wherein the cross-linkable biocompatible material comprises at least 0.5% alginate, at least 1.2 mg/ml collagen and at least 25% basement membrane preparation; preferably wherein the basement membrane preparation comprises or consists of matrigel.

11. The bioprinted tissue fiber of claim 1, wherein the first cross-linkable biocompatible material further comprises decellularized extracellular matrix and/or growth factors.

12. The bioprinted tissue fiber of claim 1, wherein the second cross-linkable biocompatible material further comprises decellularized extracellular matrix and/or growth factors.

13. The bioprinted tissue fiber of claim 1, wherein the third cross-linkable biocompatible material further comprises decellularized extracellular matrix and/or growth factors.

14. The bioprinted tissue fiber of any one of claims 1-13, wherein at least one cell type is labeled with a labeling reagent.

15. The bioprinted tissue fiber of claim 14, wherein each cell type is labeled with a different labeling reagent.

16. The bioprinted tissue fiber of any one of claims 1-15, wherein the fiber is immobilized within a printed membrane that maintains the fiber in a single plane.

17. The bioprinted tissue fiber of any one of claims 1-15, wherein the fiber is generated in a standard multi-well plate format.

18. An in vitro model of tumor cell microenvironment, comprising at least one bioprinted tissue fiber according to any one of claims 1-17.

19. A customizable theranostic device for evaluating a cancer immunotherapy in a patient in need thereof, comprising at least one bioprinted tissue fiber according to any one of claims 1-20.

20. The theranostic device of claim 19, wherein at least one of, at least two of, or each of the plurality of tumor cells, the plurality of CAF, and/or the at least one immune cell population of interest are patient-specific.

21. A method for evaluating the compatibility and/or efficacy of a candidate agent, comprising contacting at least one bioprinted fiber according to any one of claims 1-17 with the candidate agent and detecting immune cell infiltration into the core of the fiber.

22. A method of screening a candidate agent for anti-cancer activity, the method comprising: contacting a bioprinted tissue fiber according to any one of claims 1-17 with the candidate agent; and measuring an anti-cancer response in at least one cell type in the bioprinted tissue fiber; wherein an increase in the anti-cancer response in the presence of the candidate agent indicates that the candidate agent has anti-cancer activity.
